(19) United States
(12) Patent Application Publication
(10) Pub. No.: US 2016/0249817 A1
(43) Pub. Date: Sep. 1, 2016

(54) STEERABLE GUIDE WIRE WITH PRESSURE SENSOR AND METHODS OF USE

(71) Applicants: Scott T. MAZAR, Woodbury, MN (US); Peter T. KEITH, Lanesboro, MN (US); Jeffrey J. PETERS, Excelsior, MN (US); Scott R. SMITH, Chaska, MN (US)

(72) Inventors: Scott T. MAZAR, Woodbury, MN (US); Peter T. KEITH, Lanesboro, MN (US); Jeffrey J. PETERS, Excelsior, MN (US); Scott R. SMITH, Chaska, MN (US)

(21) Appl. No.: 15/009,307

(22) Filed: Jan. 28, 2016

Related U.S. Application Data
(63) Continuation of application No. 13/686,681, filed on Nov. 27, 2012, now abandoned.
(60) Provisional application No. 61/564,151, filed on Nov. 28, 2011.

(51) Int. Cl. A61B 5/0215; A61B 5/05; A61B 5/00; A61B 5/026

(52) U.S. Cl. CPC A61B 5/0215; A61B 5/026; A61B 5/05; A61B 5/6851

(57) ABSTRACT

A high performance guide wire with a pressure sensor for measuring blood pressure, may utilize a single electrical lead connected to the guide wire. An integrated circuit, powered by the single electrical connection on the guide wire, may interface with the pressure sensor, and may convert pressure information to an encoded signal. The encoded signal may be detectable in the electrical circuit, and can be used to display a pressure waveform as detected by the pressure sensor. For example, when utilized for percutaneous coronary interventions, such a guide wire can provide high quality blood pressure measurements (e.g., for fractional flow reserve), while also possessing excellent steerability and handling characteristics for navigating tortuous anatomy.

This application claims the benefit of priority under 35 U.S.C. §119(e) of U.S. Provisional Application No. 61/564,151, filed Nov. 28, 2011, the entire contents of which are incorporated herein by reference.

### TECHNICAL FIELD

Various embodiments of the present disclosure relate generally to medical devices and related methods. More specifically, particular embodiments of the present disclosure relate to a guide wire with a variable resistance pressure sensor and methods of use.

### BACKGROUND

Guide wires have become common tools in a number of medical applications. They may be used to access portions of the internal anatomy by being externally manipulated and advanced by the user to a desired position in the body. As the guide wire is manipulated, it is often visualized with the aid of x-ray fluoroscopy, endoscopy, MRI, or other imaging modalities. This visualization helps to position the guide wire. Once the guide wire is in position, other medical devices such as balloon catheters, stents, and diagnostic catheters, can be guided to a desired location by incorporation of a lumen that is advanced over the previously placed guide wire. The guide wire therefore becomes a rail to facilitate placement of the larger device within a target anatomy.

For example, in vascular applications such as angioplasty and stenting, the guide wire may first be manipulated down a vessel beyond a constricted portion of the vessel. The angioplasty/stent catheter may then be advanced over the guide wire until the working portion of the device (stent and dilation balloon) is at the constriction. Fluoroscopy combined with radiopaque portions on the guide wire and catheter may be used to view the placement of each. The balloon may be inflated to place the stent inside the blood vessel to permanently enlarge the constriction and improve blood flow.

Quite often, these blood vessels are tortuous, so the guide wires may be steerable and trackable. Steerability may be facilitated by having a “J” bend at the distal end, such that rotation imparted on the proximal end controls the orientation of the “J”, allowing for navigation through curves and branches. For rotation to be transmitted effectively, the guide wire may be nearly perfectly straight (when in its unstressed condition). Any pre-existing curvature could cause the guide wire to whip when it is rotated, rather than smoothly rotate. Further, the guide wire may be resilient to resist permanent deformation when it is flexed by being in tortuous vessels, as well as during routine handling of the guide wire. Any permanent deformation may eliminate the initial straightness qualities, again leading to whipping.

Trackability may be facilitated by having a guide wire that gradually becomes more flexible near its distal end. For example, a 0.014 inch diameter guide wire suitable for coronary artery applications may be formed with a solid core wire, with gradual tapers in the core wire near the distal end to impart greater flexibility. A highly flexible coil or sleeve extends over the distal tapered region to maintain a relatively uniform diameter while minimizing added stiffness. The resulting guide wire has significantly greater flexibility the further distal on the guide wire one measures it, when compared to the proximal portion.

A commonly used material for the core wire is 300 series stainless steel, such as type 304. This type of material has tremendous work hardening potential as it is drawn into the wire form. It is also highly biocompatible. Because it work hardens during the wire drawing process, tensile strengths in excess of 300,000 psi are often achieved. The resulting hard wire becomes highly resilient (for durability and steerability) and has a good modulus of elasticity to result in a desirable flexibility profile (for trackability) once the tapers are formed.

Recently, for some vascular applications, it has been observed that measuring blood pressure at various locations in the blood vessel prior to the treatment of the constriction can be helpful in determining if a particular constriction warrants treatment with angioplasty/stenting. Constrictions that are not severe enough to substantially impact the blood flow are best left untreated. The measurement of blood pressure upstream and downstream of the constriction can aid in this determination (to determine “fractional flow reserve” or FFR). Therefore, various pressure sensors have been incorporated into certain guide wires near their distal ends, adding functionality to those guide wires.

Certain pressure sensing guide wires use a piezo-resistive pressure sensor in either a Wheatstone bridge configuration or a half Wheatstone bridge configuration near the distal end of the guide wire. Multiple insulated lead wires may be needed to connect to the various nodes on the sensor. To facilitate multiple lead wires, the guide wire may be hollow, allowing lead wires to occupy the hollow space. The lead wires may then be connected between the sensor and a connector on the opposing end of the guide wire. Wiring and circuitry within the guide wire may be insulated, as any current leakage could be erroneously interpreted as a change in pressure.

The proximal, stiffer portion of these pressure sensing guide wires may be formed with a hypotube with the lead wires extending inside. Using a hypotube, a readily available component, greatly compromises guide wire steerability. Hypotubes are not nearly as resilient, and have different flexibility, than guide wires made from solid wire.

Furthermore, to facilitate electrical connection to the multiple lead wires running inside the hypotube, a series of connecting rings may be fabricated at the proximal end. A proximal connector may be connected to these rings to connect the guide wire to an external power source and monitor. Whenever pressure data is being measured, this connector may be in place. However, the presence of this connector inhibits the rotation and advancement of the wire needed during navigation within the body. Quite often it may be removed during the vascular navigation steps, further complicating the procedure.

There is a need for guide wires that are capable of measuring pressure, but possess higher performance characteristics than other designs.

### SUMMARY

According to one aspect of the presently disclosed embodiments, an elongate intravascular guide wire may include an electrically conductive core wire connected to a sensor housing. The guide wire may also include a pressure sensor and an integrated circuit located within the sensor housing. The guide wire may also include at least one return electrode connected to the integrated circuit. The integrated circuit, pressure sensor, and return electrode may be electrically connected to form part of a primary electrical circuit. The guide wire may also include an electrical connector disposed on a proximal portion of the core wire. The electrical connector may be electrically connected to the core wire. The core wire may be configured to deliver electrical power to the integrated circuit from the connector. The integrated circuit may be configured to transmit electrical information.

According to another aspect of the presently disclosed embodiments, a system for measuring intravascular blood pressure within a mammal may include an elongate guide wire. The elongate guide wire may include a pressure sensor at a distal portion of the core wire, the pressure sensor being configured to sense blood pressure. The guide wire may also include an integrated circuit at the distal portion of the core wire, the integrated circuit being electrically connected to the core wire. The system may also include a steering device in electrical communication with the core wire. The system may also include a power source connected to the steering device. The system may also include a guiding catheter surrounding at least a portion of the guide wire. The integrated circuit may operate with the pressure sensor to convert the sensed pressure to an information encoded signal sent along the guide wire.

According to another aspect of the presently disclosed embodiments, a pressure sensor circuit for an electrically conductive guide wire may include a piezo-resistive pressure sensor having at least one resistance element that changes resistance in response to changes in applied pressure. The circuit may also include an integrated circuit electrically connected to the pressure sensor and powered by alternating electrical current delivered via the guide wire. The integrated circuit may be configured to convert the resistance to a pulse. The pulse may cause a signal driver to impose a first voltage change on the guide wire at a time corresponding to a first pulse transition and to impose a second voltage transition on the guide wire at a time corresponding to a second pulse transition. The circuit may also include a data processor external to the guide wire. The data processor may be configured to convert timing between the first voltage transition and second voltage transition into a recovered time interval, and convert the recovered time interval into an indication of pressure.

According to another aspect of the presently disclosed embodiments, a method for measuring blood pressure in a patient may include advancing a guide wire to a desired position in the patient's blood vessel. The guide wire may include a pressure sensor and an integrated circuit at a distal portion of the guide wire. The integrated circuit may be configured to send an information encoded signal representative of blood pressure through the guide wire. The method may also include operating a power source electrically connected to a connector in electrical communication with the guide wire, to send electrical current along the guide wire. The method may also include measuring the pressure at the desired position.

According to another aspect of the present disclosed embodiments, a system for measuring blood pressure may include a power supply. The system may also include a guide wire assembly operatively coupled to the power supply. The guide wire assembly may include an electrically conductive core wire connected to a sensor housing. The guide wire assembly may also include a pressure sensor and an integrated circuit located within the sensor housing. The guide wire assembly may also include at least one return electrode connected to the integrated circuit. The integrated circuit, pressure sensor, and return electrode may be electrically connected to form part of a primary electrical circuit. The guide wire assembly may also include an electrical connector disposed on a proximal portion of the core wire. The electrical connector may be electrically connected to the core wire and the power supply. The core wire may be configured to deliver electrical power to the integrated circuit from the connector. The integrated circuit may be configured to transmit electrical information. The system may also include a guide catheter configured to receive at least a portion of the guide wire assembly. The guide catheter may include a conductive braid configured to receive the electrical information from the integrated circuit. The guide catheter may also include a guide catheter connector operatively coupled to the braid. The guide catheter connector may be configured to receive the electrical information from the braid, and deliver the electrical information to an output device.

According to another aspect of the presently disclosed embodiments, a method for determining intracorporeal pressure in a patient may include inserting a transducer, and an integrated circuit operatively coupled to the transducer, into a portion of a body for detecting the pressure in the portion of the body. The conductor may be operatively coupled to the integrated circuit. The method may also include directing power from a power supply to the conductor. The power may be in the form of an alternating current. The method may also include delivering the alternating current to the integrated circuit and the transducer through the conductor. The method may also include modulating the alternating current with at least one of the transducer and the integrated circuit, in response to the pressure detected.

According to another aspect of the presently disclosed embodiments, a connector configured to steer a guide wire may include an elongate body defining a lumen. The lumen may be configured to receive the guide wire. The connector may also include a clamp configured to mechanically grip the guide wire. The connector may also include an electrical connection coupled to the clamp. The electrical connection may be configured to deliver power from a power source through the clamp and to the guide wire.

According to another aspect of the present disclosed embodiments, a pressure sensing guide wire assembly, for sensing blood pressure in a patient's body, may include a guide wire including a distal portion and a proximal portion. The guide wire assembly may also include a sensor assembly operatively coupled to the distal portion of the guide wire. The sensor assembly may include an integrated circuit operatively coupled to the guide wire. The sensor assembly may also included a sensor operatively coupled to the integrated circuit. The sensor may be configured to sense pressure, and the integrated circuit may be configured to convert the sensed pressure into encoded information. The sensor assembly may also include a sensor housing configured to receive the integrated circuit and the pressure sensor.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is a side view illustration of an exemplary pressure sensing guide wire, consistent with embodiments of the present disclosure.

FIG. 2 is a side view illustration of another exemplary embodiment of a pressure sensing guide wire, consistent with embodiments of the present disclosure.

FIG. 3 is a schematic view illustration of an exemplary system including a pressure sensing guide wire, in use within a patient, consistent with embodiments of the present disclosure.

FIG. 4 is a perspective view illustration of a portion of an exemplary pressure sensing guide wire, in a region of a pressure sensor, consistent with embodiments of the present disclosure.

FIG. 5 is an exploded perspective view illustration of an exemplary pressure sensing guide wire, in a region of a pressure sensor, showing internal electrical components, consistent with embodiments of the present disclosure.

FIG. 6 is a block diagram illustration showing electrical functional elements of an exemplary pressure sensing guide wire, consistent with embodiments of the present disclosure.

FIGS. 7a and 7b are graphical illustrations showing exemplary waveforms of portions of the block diagram shown in FIG. 6, consistent with embodiments of the present disclosure.

FIGS. 8a and 8b are graphical illustrations showing exemplary waveforms of other portions of the block diagram shown in FIG. 6, consistent with embodiments of the present disclosure.

FIGS. 9a and 9b are graphical illustrations showing waveforms of other portions of the block diagram shown in FIG. 6, consistent with embodiments of the present disclosure.

FIGS. 10a and 10b are graphical illustrations showing waveforms of other portions of the block diagram shown in FIG. 6, consistent with embodiments of the present disclosure.

FIG. 11a is a perspective view illustration of a portion of an exemplary pressure sensing guide wire, in a region of a pressure sensor, consistent with embodiments of the present disclosure.

FIG. 11b is a longitudinal section side view illustration of the portion of the exemplary pressure sensing guide wire, in the region of the pressure sensor, shown in FIG. 11a, consistent with embodiments of the present disclosure.

FIG. 12 is a top view illustration of an integrated circuit, consistent with embodiments of the present disclosure.

FIG. 13a is a perspective view illustration of a portion of an exemplary pressure sensing guide wire, in a region of a pressure sensor, consistent with embodiments of the present disclosure.

FIG. 13b is a perspective longitudinal section view illustration of a cross-section of the portion of the exemplary pressure sensing guide wire, in the region of the pressure sensor, shown in FIG. 13a, consistent with embodiments of the present disclosure.

FIG. 14a is a perspective view illustration of a portion of an exemplary pressure sensing guide wire, in a region of a pressure sensor, consistent with embodiments of the present disclosure.

FIG. 14b is another perspective view illustration of a portion of the exemplary pressure sensing guide wire, in the region of the pressure sensor, shown in FIG. 14a, consistent with embodiments of the present disclosure.

FIG. 15a is a perspective view illustration of a portion of an exemplary pressure sensing guide wire, in a region of a pressure sensor, consistent with embodiments of the present disclosure.

FIG. 15b is a longitudinal section side view illustration of the portion of the exemplary pressure sensing guide wire, in the region of the pressure sensor, shown in FIG. 15a, consistent with embodiments of the present disclosure.

FIG. 16a is a side view illustration of an exemplary connector, consistent with embodiments of the present disclosure.

FIG. 16b is a side view illustration of a cross-section of the exemplary connector, shown in FIG. 16a, consistent with embodiments of the present disclosure.

FIG. 17 is a schematic view illustration of an exemplary system including a pressure sensing guide wire, in use within a patient, consistent with embodiments of the present disclosure.

FIG. 18 is a schematic view illustration of an exemplary system including a pressure sensing guide wire, consistent with embodiments of the present disclosure.

FIG. 19 is a schematic view illustration of an exemplary system including a pressure sensing guide wire, in use within a patient, consistent with embodiments of the present disclosure.

FIG. 20A is a perspective view illustration of an exemplary guide catheter clip, consistent with embodiments of the present disclosure.

FIG. 20B is an axial section front view illustration of an exemplary guide catheter clip, consistent with embodiments of the present disclosure.

FIG. 20C is a longitudinal section perspective view illustration of the exemplary guide catheter clip, shown in FIG. 20B, consistent with embodiments of the present disclosure.

FIGS. 21A-21D are graphical illustrations showing exemplary resistance modulation and digital encoding, consistent with embodiments of the present disclosure.

FIG. 22A is a side view illustration of exemplary electronic components, consistent with embodiments of the present disclosure.

FIG. 22B is a top view illustration of exemplary electronic components, consistent with embodiments of the present disclosure.

FIG. 22C is a side view illustration of a cross-section of exemplary electronic components, consistent with embodiments of the present disclosure.

FIG. 23 is a graphical illustration showing exemplary waveforms, consistent with embodiments of the present disclosure.

FIG. 24 is a graphical illustration showing exemplary waveforms, consistent with embodiments of the present disclosure.

### DETAILED DESCRIPTION

Reference will now be made in detail to the exemplary embodiments of the disclosure, examples of which are illustrated in the accompanying drawings. Wherever possible, the same reference numbers will be used throughout the drawings to refer to the same or like parts.

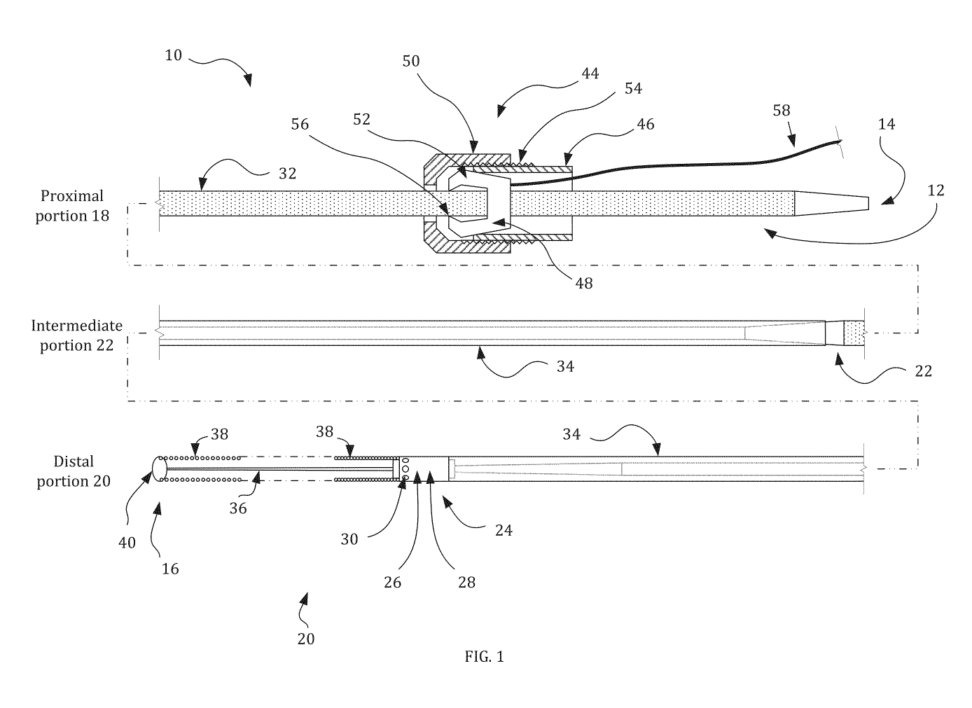

Exemplary features of one embodiment of the guide wire 10 of the present disclosure is shown in FIG. 1. This embodiment may be a steerable coronary guide wire, however the same principles described in connection with this and all embodiments herein could be applied to other guide wire embodiments as well.

Guide wire 10 includes a core wire 12. Core wire 12 may be a solid wire, and may be fabricated from a conductive material such as stainless steel (e.g., hard drawn type 304) or other biocompatible conductive materials. A preferred material is type 304 that is hardened from the drawing process to a tensile strength of 300,000 psi or greater. This results in a core wire which is resilient, contributing to steerability and durability of the guide wire during a medical procedure. Alternative materials are also contemplated, such as nickel titanium alloy (nitinol), cobalt chromium alloys, other grades of stainless steel, or other resilient high strength biocompatible materials, as well as various combinations thereof. While a solid wire is preferred for the core wire 12, a hollow tube is also contemplated as other features can still be realized with a hollow tube as the core wire 12.

Guide wire 10 has a proximal end 14 and a distal end 16, between which are three portions: (i) a proximal portion 18 which extends from proximal end 14 to a (ii) tapering intermediate portion 22, where the core wire 12 has reduced diameter to impart greater flexibility for navigating curves such as the curves in coronary blood vessels; and (iii) a distal portion 20. At the distal end of the intermediate portion 22 is the sensor housing 24. Distal to the sensor housing 24 is the distal portion 20. Distal portion 20 is formed of a distal core wire 36, surrounded by a distal coil 38, and a distal tip 40.

Core wire 12 terminates at the proximal end of the sensor housing 24, freeing up space within the sensor housing 24 for a transducer or sensor 26 and an integrated circuit (IC) 28 inside (not visible in this view). One or more access windows 30 formed in sensor housing 24 allow for blood pressure to be conveyed to the sensor 26.

Core wire 12 may have a tapering feature adjacent the proximal end 14, to facilitate adding an extension wire to extend the length of the guide wire, if “over the wire” catheters are needed to be exchanged over the guide wire 10. For substantially the entire length of the proximal portion 18 of core wire 12, a lubricious coating 32 such as PTFE may be applied. Such lubricious coating 32 further provides an electrical insulation to the core wire 12.

As mentioned, the intermediate portion 22 may have diametric tapering of the core wire 12 to impart greater flexibility. Such tapering may be formed by centerless grinding. To maintain a uniform outer diameter, a sleeve 34 may surround the core wire 12 in the reduced diameter regions. Such sleeve 34 may additionally serve to insulate the core wire 12 in this portion.

Core wire 12 is attached to the proximal end of the sensor housing 24 in a manner that mechanically connects them, but does not allow for electrical communication to the sensor housing 24. Sensor housing 24 could be formed of a non-conductive material such as ceramic or rigid polymer. In this case, any suitable mechanical bonding, such as adhesive bonding, can be used. Alternatively, sensor housing 24 could be metallic (and conductive). In this case, an insulating bonding layer, such as a ceramic coating, could be applied to the outside of the core wire 12 to facilitate mechanical bonding, such as adhesive bonding, without electrical communication to the sensor housing 24.

Inside sensor housing 24, the distal end of the core wire 12 is electrically connected to the IC 28, as will be described in further detail below. The IC 28 is powered by current delivered down the core wire 12 near the proximal end, from an external power source via a connector 44, also described in more detail below. The distal core wire 36 is mechanically secured to the distal end of the sensor housing 24, using similar techniques as the attachment of the distal end of the core wire 12 to the proximal end of the sensor housing 24.

To form a complete primary electrical circuit 114, the IC 28 is in electrically connected to the distal core wire 36 and/or the distal coil 38. If the distal tip 40 of the guide wire 10 is a weld, solder, or braze joint, then distal coil 38 and distal core wire 36 will both be in electrical communication with the IC 28. Since the distal portion 20 of the guide wire 10 is in the electrically conductive bloodstream, the distal coil 38 serves as a return electrode. A grounding pad 42, such as an EKG pad affixed to the patient, can be wired back to an external power source, thus completing the primary circuit 114.

The input power required to operate the sensor and IC circuitry will be relatively small, and current leakage concerns into the patient will be mitigated. This is particularly true if an alternating current waveform at relatively high frequency is utilized to power the IC and sensor circuitry.

Electrical power is delivered to the guide wire 10 via a connector 44. Connector may operate as the torque device or steering device which may be used to manipulate steerable guide wire 10.

One embodiment of connector 44 includes a finger grip 46, a clamp 48, and a nut 50. The clamp 48 includes tangs 52 that compress onto the guide wire 10 when the nut 50 is tightened against the finger grip 46 via threads 54. To deliver electrical power, tangs 52 further include one or more sharpened tips 56, which can penetrate the insulative lubricious coating 32 to make contact with the conductive core wire 12. Clamp 48 is conductive, and is connected to a lead wire 58 which is connected to the power source. Lead wire 58 may be connected to the clamp 48 directly by means such as solder, or may be connected via rotary connection, which allows the connector 44 to rotate and steer the guide wire 10 without causing the lead wire to twist around the guide wire 10 proximal of the connector 44.

In an alternative embodiment of the connector 44, the tangs 52 do not include sharpened tips, but rather may be simply compressed against the lubricious coating until they are in close proximity to the conductive core wire 12. The lubricious coating acts as a dielectric, and the close proximity of the conductive tangs 52 to the core wire 12 results in a capacitive coupling. As long as the input drive signals on the guide wire 10 are alternating, these signals can pass through to the core wire 12 capacitively without direct electrical contact. This embodiment avoids the mechanical piercing and disruption to the lubricious coating 32.

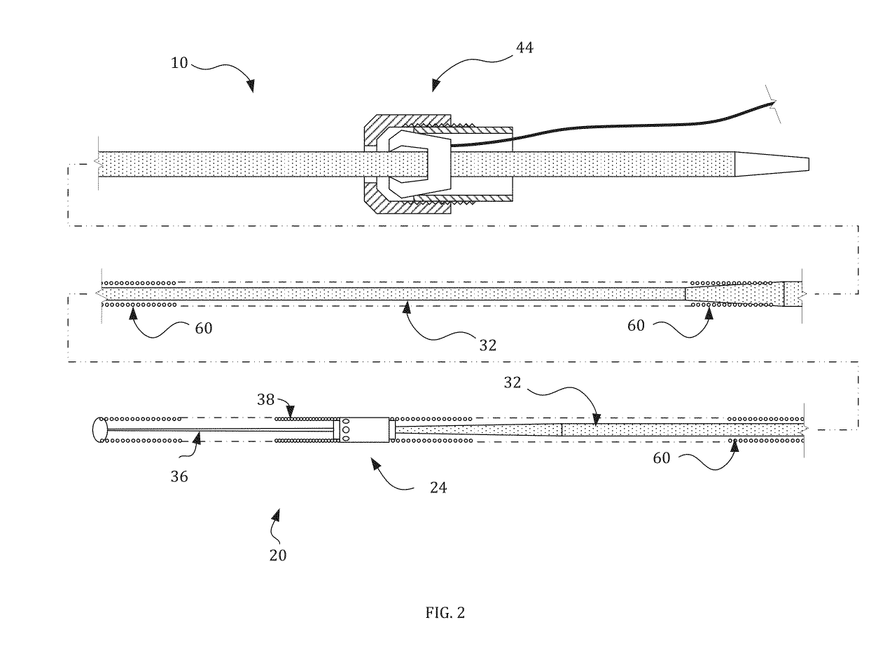

FIG. 2 illustrates an alternative embodiment, in which a proximal coil 60 is utilized as the return electrode, instead of or in addition to the distal coil 38 of the embodiment of FIG. 1.

Rather than a sleeve, metallic proximal coil 60 is utilized to maintain the uniform diameter of the guide wire 10. In this embodiment, an insulative coating such as PTFE or ceramic may be applied to the surface of the reduced diameter portions of the intermediate portion 22 of guide wire 10. The proximal coil 60 and core wire 12 are secured to the proximal end of the sensor housing in such a way as to provide for electrical connection between the IC 28 and the proximal coil 60, described in further detail below. This embodiment provides for greater surface area for the return electrode, as proximal coil 60 can typically be longer than distal coil 38. This is because in certain embodiments, it may be desirable for the sensor 26 to be near the distal tip 40 of guide wire 10, thus resulting in a relatively short length of the distal portion 20.

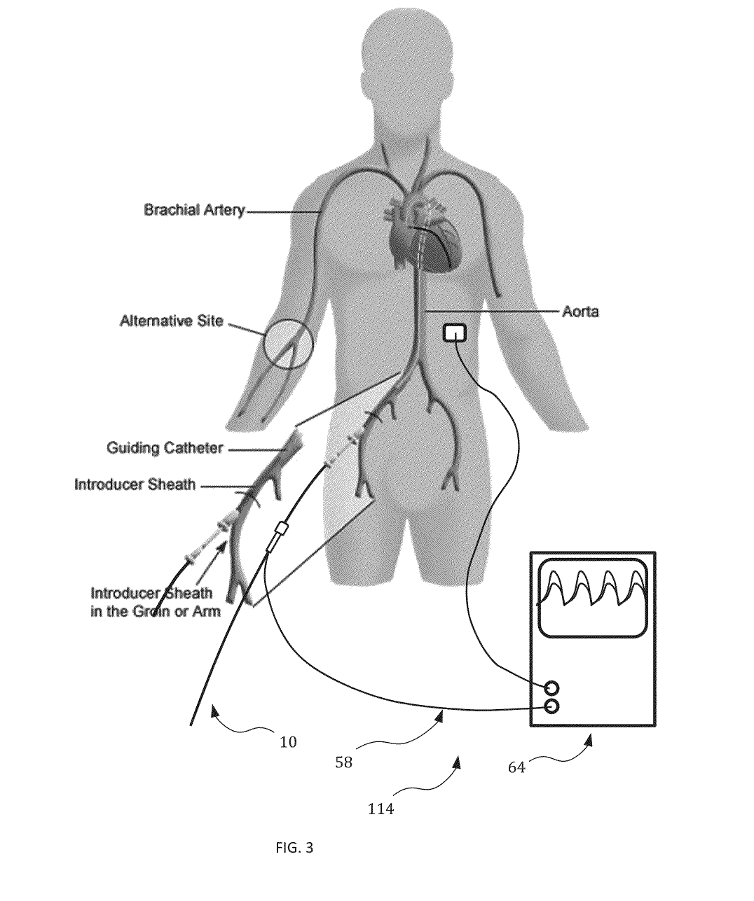

FIG. 3 depicts an embodiment of the guide wire 10 in use in a human patient, and illustrates the primary circuit 114. In this embodiment, guide wire 10 is a steerable coronary guide wire used in conjunction with a potential stent implantation. A guiding catheter 62 is present in the patient, extending from an access site (in this case, the femoral artery), through the aorta, and engaging the ostium of a coronary blood vessel. The guide wire 10 extends through the guiding catheter 62 and is advanced and steered down the desired coronary artery, beyond one or more narrowed or constricted areas that may be targets for angioplasty and stenting. In addition to establishing a conduit from the femoral artery access site, the guiding catheter 62 further insulates the guide wire 10 from the patient's body, and greatly minimizes capacitive losses along the length of the guide wire 10. The guiding catheter 62 may also include a pressure sensor (not shown) for measuring pressure upstream from the narrowed or constricted area. For example, the guiding catheter 62 may measure aortic blood pressure in the aorta using a pressure sensor at a distal end of the guiding catheter 62.

The guide wire 10 is connected via the connector 44 and lead wire 58 to the power source 64. A grounding pad 42, like an EKG type pad, attached to the patient, is also connected to the power source 64 to complete the primary electrical circuit 114. Power source 64 may also include a display 66. Electrical signals travel down the guide wire 10 and are converted into a pressure waveform as will be further described below. Additional signal inputs can be traced as well on the display 66 such as the aortic blood pressure, typically measured via the guiding catheter 62. Display 66 may also be a separate component.

As the guide wire 10 is positioned in various locations, the blood pressure can be measured and compared to the aortic blood pressure. For example, if the blood pressure as measured by the guide wire 10 at a location distal to a narrowing is significantly lower than the aortic blood pressure, the narrowing may be determined to be critical enough to warrant dilation and stenting. However, if the distal blood pressure is not substantially lower than the aortic blood pressure, the narrowing may not be dilated and stented, thus saving substantial costs and avoiding unnecessary risks to the patient of having a permanent stent positioned in that narrowing. One way to compare the aortic blood pressure to the distal blood pressure is to determine a fractional flow reserve (“FFR”). FFR may be a ratio of distal pressure to aortic pressure. If FFR crosses a predetermined threshold value, it may indicate that a critical narrowing or constriction is present.

If stent placement is desired, the connector 44 is removed, and the stent delivery catheter (not shown) is advanced over the guide wire 10 to the site of narrowing. Because the connector 44 is also the steering device, and not a separate device as in other pressure guide wires, only this one device needs to be removed from the guide wire 10 to facilitate loading and advancement of the stent delivery catheter.

FIG. 4 is an enlarged view of the part of the guide wire 10 incorporating the sensor 26 and IC 28. A portion of the distal core wire 36, a portion of the distal coil 38, a portion of the proximal core wire 12 and a portion of the proximal coil 60 are shown, all connected to the sensor housing 24.

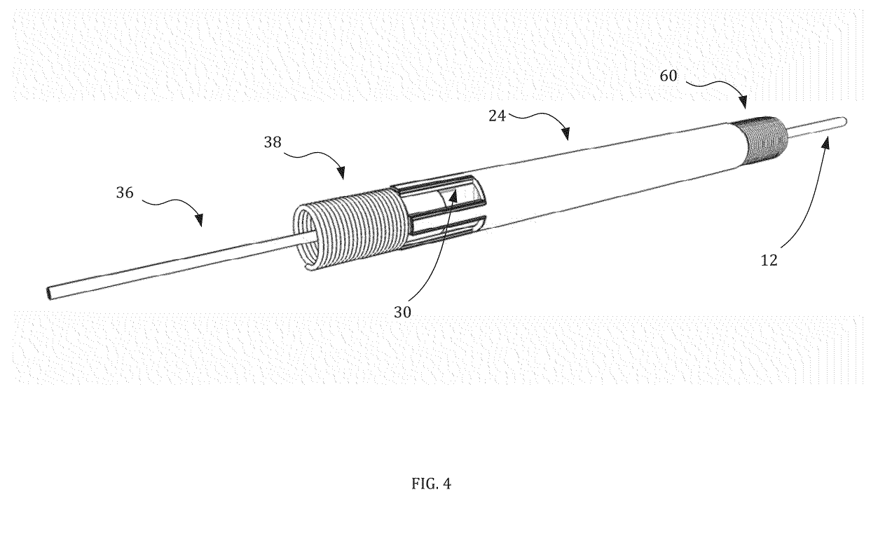

FIG. 5 is an exploded view of FIG. 4, showing an embodiment of the guide wire 10 showing the sensor 26 and IC 28, which would reside within the sensor housing 24. Sensor 26, which may be a piezo-resistive sensor with at least one or more resistors etched in a deflectable silicon wafer, as well as one or more resistors etched in a non-deflectable portion of sensor (which may serve as calibration resistors), is electrically connected the IC 28, and will be further described below. The variable resistors change their resistance in response to mechanical deformations caused by changes in local pressure applied to the sensor 26. IC 28, which is powered by the power source 64, sends electrical signals to the sensor 26. The IC 28 incorporates a logic module which essentially responds to the variations in resistance by converting the resistance changes to time based voltage impulses that are applied back onto the guide wire 10 and received and relayed by the display 66. A similar “resistance to time” logic circuit is described in Jeong, et al., “A low-cost resistance-to-time converter for resistive bridge sensors”, in The 23^{rd} International Technical Conference on Circuits/Systems, Computers and Communications (ITC-CSCC 2008), pp. 1137-1140.

The one or more resistors that are incorporated into the sensor 26 are electrically connected to the IC 28. The IC 28, which is powered via the core wire 12, has an electrical connection as shown. If the proximal coil 60 serves as the return electrode, another electrical connection to the proximal connection 68 provides electrical continuity to the proximal coil 60. An insulative coating on the outside of the distal end of the core wire 12 prevents a short from the core wire 12 to the proximal coil 60, while providing for a mechanical joining means between the core wire 12, proximal coil 60 and proximal end of sensor housing 24. A distal connection 70 provides for a mechanical joining or joint between the distal end of sensor housing 24 and the distal core wire 36 and distal coil 38.

To minimize mechanical artifact to the sensor, due to bending forces acting on the guide wire, the sensor 26 resides within the sensor housing 24. In one embodiment, and as shown in FIG. 5, the sensor 26 and IC 28 are not mounted to the core wire 12, but essentially float within the housing. This arrangement further minimizes artifactual stresses induced by mounting the sensor to a substrate. Alternative embodiments may extend the core wire 12 or some other mechanical structure into the sensor housing 24 for attachment of the sensor 26.

To further isolate the sensor 26 from external influences other than fluid pressure, the interior of the housing may be fluid filled, with a highly wettable priming fluid 74 such as Fluorinert. To keep the priming fluid 74 in place, a gel or soft elastomeric plug 72 is positioned within the access windows 30. Pressure transmits through the soft plug 72 and priming fluid to the sensor 26.

In an alternative embodiment, the interior of the sensor housing 24 is kept open and dry until guide wire 10 is to be used. At that time, the interior can be prepped using a liquid such as saline, which can be applied via the access window(s) 30. To further facilitate priming, one or more additional access window(s) can be positioned closer to the proximal end of the sensor housing 24. To prevent shorting of the sensor 26 and IC 28, the internal components within the sensor housing 24 may be insulated, such as with a parylene coating.

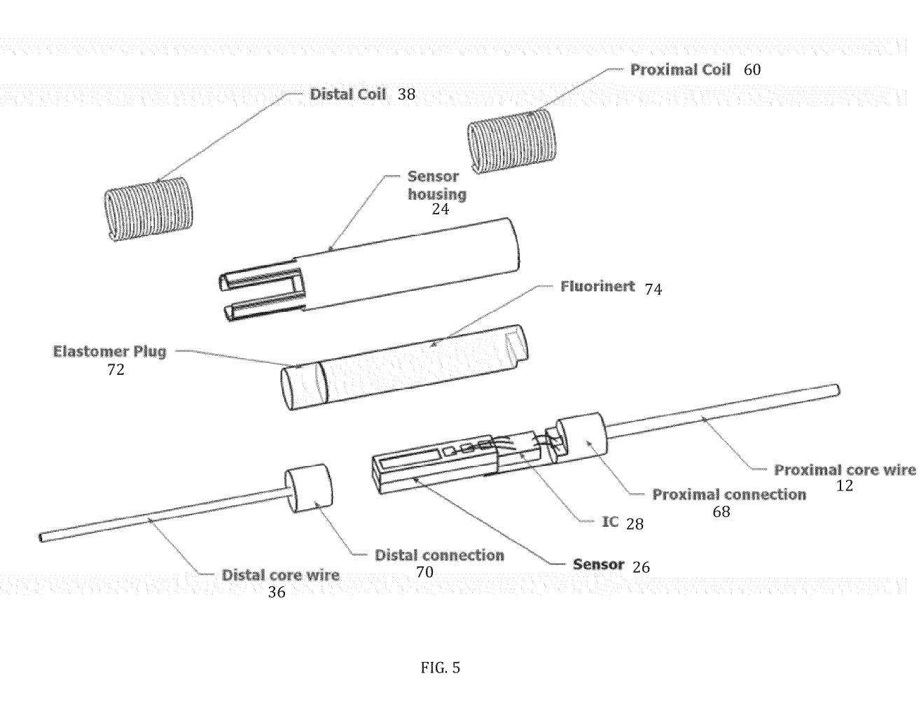

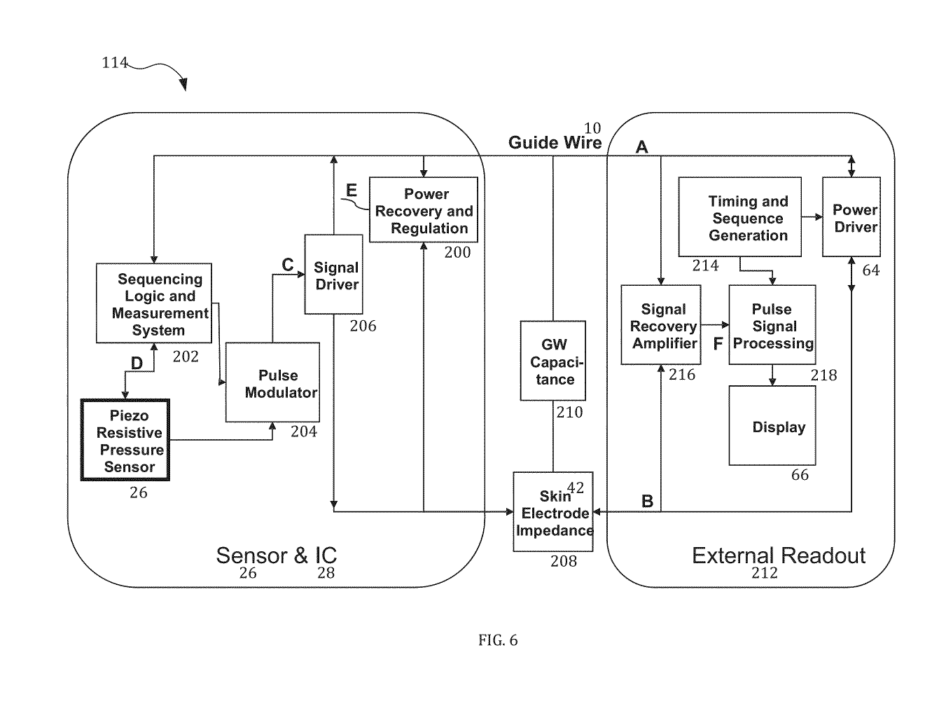

The electrical components of one embodiment of the present disclosure are shown in FIG. 6, and further described in detail below. The large box on the left (sensor and IC 26 and 28) includes the pressure sensor 26 which includes the variable resistance element(s) and the control resistance element(s), as well as the functional components that are part of the IC 28. The sensor 26 and IC 28 are located within the sensor housing 24.

The large box on the right (external readout 212) includes the power source 64 which sends alternating current down the guide wire 10 to power the IC 28, a display 66 which displays the desired output, e.g. a pressure waveform, after the impulses imparted to the wire 10 by the IC 28 are processed back into pressure information.

The primary circuit 114 is driven by the power source 64, which sends an input drive signal down the guide wire 10. The input drive signal may be current driven, in which case the drive signal will have a drive signal current waveform 100i. The impedance characteristics of all the electrical components in the primary circuit 114 will influence the resultant voltage waveform 100v that can be measured at the power source 64. Various alterations or modulations to the voltage 100v as controlled by the IC 28 may be encoded as pressure data from the sensor 26, and then decoded to result in a processed pressure waveform 360, as will be further described below.

The IC 28 includes components 200 that recover power delivered through the guide wire 10 in the form of a symmetrically oscillating current square wave 100i to provide regulated voltage to internal sensor components. Recovered power operates sequencing logic 202, a piezo resistive pressure sensor 26 and pressure sensor measurement circuits, a pulse modulator 204 and a signal driver 206. A function of IC 28 is to convert piezo resistive pressure sensor output into pulses of a duration related to the pressure and to transmit those pulses to external readout 212 by signal driver 206. The pulses can represent pressure sensor output in a number of forms. For example, the pulses may be pulse width modulated in relation to pressure. Alternatively, sensor signals may be digitized and the pulses may represent the digitized data bits.

The power recovery and regulation block 200 may include a bridge rectifier and two stages of regulation. The first stage of regulation is a shunt regulator that clamps the peak voltage output from the rectifier. A clamped voltage may be used since parasitic resistance in the return path requires the external power driving source to be a current source. A current source will drive current by allowing voltage to vary until some externally imposed voltage limit is reached, possibly even to the point of delivering excess voltage to electrical components. In this case, that limit is defined by a shunt regulator.

The output of the shunt regulator feeds a small capacitance at the input of a second stage of regulation. The second stage regulator is a series regulator that smooths any remaining voltage variations from the shunt regulator and capacitor.

Power is delivered to the sensor as a constantly reversing current. The current is set by an external power driving source and is at a frequency and amplitude that are compliant with the AAMI ES1 electrical safety standard for what the standard refers to as risk current. To make as much current available to the sensor as possible and to minimize the size of the filter capacitor across the shunt regulator, a high frequency driving current is preferred. In this case, a 100 KHz square wave is used to drive the sensor power recovery and regulation circuit allowing up to 1 mA peak current at the sensor.

The AAMI ES1 specification limits current at DC to a value of less than 10 uA. Since the system uses the body as a return path, it may be preferable for the DC current component of the driving waveform to be symmetrical, so that the average DC current is as close to zero as possible.

Sequencing of internal processes in the sensor 26 can be based on timing derived from the drive power waveform. Both the rising and falling edge of the drive waveform can be used to initiate measurements. Sequencing logic 202 controls data conversion of the output signal from piezo resistive sensor 26, pulse modulation timing and if needed, blanking to mask transient intermediate signals. Alternatively, timing can be derived from an oscillator in the IC 28 itself.

The sensor 26 may be a piezo resistive sensor and may include either a full Wheatstone bridge or a half Wheatstone bridge. The half Wheatstone bridge is completed by the addition of fixed resistors. For example, in a Pulse Width Modulated (PWM) embodiment, a forced bridge imbalance is used to ensure that the PWM output never reaches zero or negative pulse time. This imbalance is created either by the addition of a series resistance in one side of the bridge or a series resistance in both sides of the bridge. Forced bridge imbalance output is taken from opposing ends of the imbalance resistors or one end of a single imbalance resistor.

Regulated voltage is applied to the Wheatstone bridge and external pressure creates an output voltage imbalance in addition to any forced bridge imbalance. Other embodiments may make use of different types of sensors, for example, capacitive sensors.

Pressure data gathered by the IC 28 may be communicated in many ways, such as modulation of the power waveform. The following is but one example. Output from the Piezo Resistive Sensor 26 (PRS) is digitized in the pulse modulator 204, also called a converter. Pulses may be generated by a Pulse Width Modulator (PWM) or other digitizing system, such as an Analog to Digital Converter (ADC).

In the case of a PWM, a pulse is generated with a time duration that is related to the PRS output. The pulse duration may include a constant time factor plus a variable time factor, where the variable time factor varies in proportion to the PRS input pressure signal. Pulses from an ADC can either be present or absent, or can be short pulses and long pulses to represent logical 1's or 0's.

In one embodiment, to generate a PWM signal, a ramp waveform is generated that is applied to the inverting inputs of two comparators. The non-inverting inputs of the comparators are respectively connected to each side of the Wheatstone bridge. As the ramp waveform increases in voltage, a first comparator will switch output state indicating a first timing edge. As the waveform continues to rise, a second comparator will switch output state indicating a second timing edge. To generate a pulse, the outputs of the comparators are exclusive OR'ed together generating a pulse that is present only when the comparator outputs differ. The process can be repeated in reverse by a downward ramping waveform of the same voltage slope as the upward ramping waveform. The pulse duration of a system using a 100 KHz power drive signal can range between 1 and 4 microseconds.

An alternative to using two comparators that may have significantly different offset voltages is to use a single comparator that is switched between each of the sides of the Wheatstone bridge. Logic would control comparator input switching and convert the comparator output into equivalent output signals as the two-comparator embodiment.

To control sensitivity, ensure bounded voltage limits on the ramp waveform and control the location of the PWM pulse signal in relation to the power drive waveform, the outputs of the comparators with small added delays can be used to control the ramp generator.

Pulses from the pulse modulator 204 may be suitable for direct modulation of the voltage on the guide wire 10 or may require additional power or wave shaping. The signal driver 206 converts logic signals from the pulse modulator 204 into signals that can be received by the external readout.

One form of signal driver 206 signal conversion is to high pass filter PM pulses, converting them into spike-like impulses. These impulses can then be separated from the drive waveform by similar high pass filtering located at the external readout equipment.

The guide wire 10 and skin electrode connection 42 form the electrical connections between the pressure sensor 26 and IC 28 located in the body and external readout equipment 212. The metallic guide wire 10 forms a direct electrical connection to power the in-body circuitry. The return path may include, for example, the proximal coil 60 of the guide wire 10. The proximal coil 60 may be used to reduce current density to the maximum extent possible because of its greater length compared to the distal coil 38. Current returned through the proximal coil is 60 collected by an electrode 208 attached to the skin, which may include, for example, a small ECG electrode or a large defibrillation patch.

The guide wire 10 may include a conductive metal core 12 covered by a thin layer of insulating material 32. When in the body, the insulation 32 forms the dielectric of a capacitor with one plate of the capacitor made of the conductive wire 12, and the other plate made of the surrounding tissue and conductive fluids of the body.

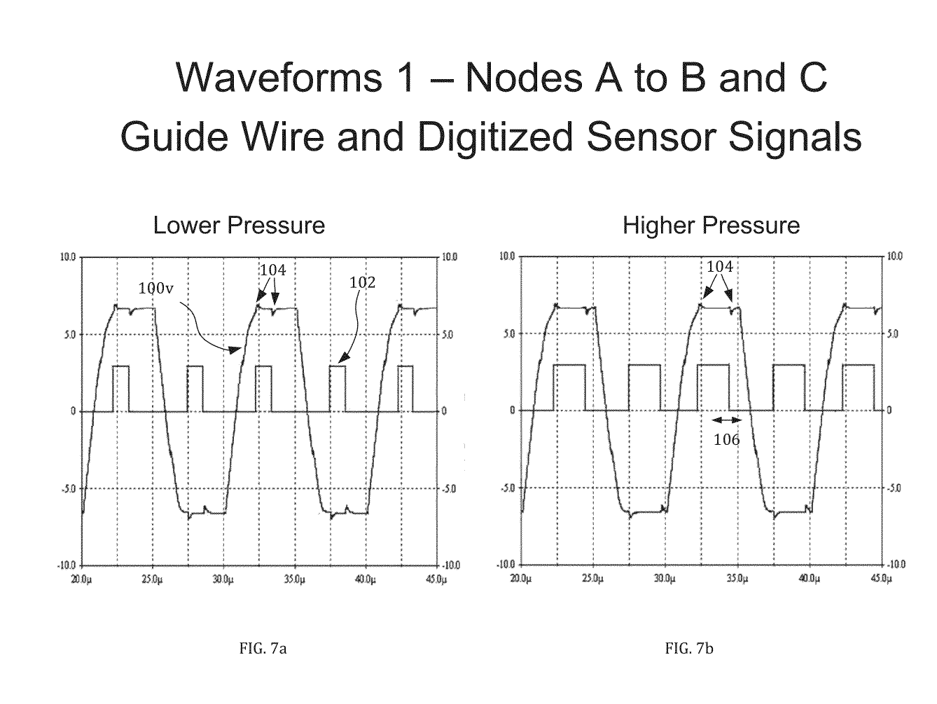

Capacitance 210 from the guide wire 10 dielectric insulation 32 causes displacement currents to flow when driven by a square wave current. These displacement currents cause the voltage 100v on the guide wire 10 to have transitions with finite voltage slope (as seen, for example, in FIGS. 7a and 7b).

The Skin Electrode Connection (SEC) 208 may include an electrode placed on the skin, such as the electrode 42. The electrode 42 may include a small ECG patch to a large defibrillation electrode. A function of the SEC 208 is to collect return current from the proximal coil 60 of the guide wire 10. From an electrical standpoint, the SEC 208 includes a series resistance paralleled by a capacitance.

The external readout 212 may be made up of subsystems used to power the sensor 26 and IC 28 and to recover and display data measured by the sensor 26.

Timing and sequence generation creates control waveforms that sequence a power driver 64 and aid in signal recovery. The operating frequency is set high enough that sufficient “risk current”, as defined by the AAMI ES1 safety specification, is available to operate sensor circuits. A preferred frequency of operation is 100 KHz. It is also preferable that the drive current is as symmetrical as possible to minimize the DC component to a “risk current” value that meets the AAMI ES1 electrical safety specification.

The power driver (or power source) 64 is a controlled current source that supplies current to the guide wire 10. The current source may include a high voltage compliance current source with the ability to switch polarity to form a square wave current of at least 100 KHz.

The signal recovery amplifier 216 may include a high pass filter to pass data impulses generated by the sensor/IC signal driver 206 and to block guide wire 10 voltage variations caused by the power driver 64. The high passed signals are amplified and detected by, for example, a Schmitt trigger. The output of the Schmitt trigger represents an analog of the pulse generated by the sensor/IC 26/28. The Schmitt trigger output will vary in pulse width and pulse presence in direct relation to pulses generated at the sensor/IC 26/28. Alternatively, a comparator can detect the voltage variations in direct relation to pulses generated at the sensor/IC 26/28.

The pulse output of the Signal Recovery Amplifier 216 can be directly measured and converted into a representation of pressure. Using either direct time measurement or a pulse integration and digitization method can provide at least 100 measurement levels or 7 bits of resolution, in a pulse width modulation scheme.

The pulse output can also be processed to reduce signal noise and increase resolution by performing pulse width or signal averaging.

The benefit of signal averaging is a substantial improvement in resolution. For example, if the drive waveform has a frequency of 100 KHz, a pulse width modulator that uses both an upward and downward slope to generate a pulse will generate data at a rate of 200k samples per second. If the end bandwidth is intended to be 200 Hz, the system over samples the sensor at by 1000 times per output reading. Noise can be reduced by the square root of the over sample rate or about 32 times. Signal averaging in this case provides an additional 5 bits of signal resolution in addition to the at least 7 bits of resolution from pulse to pulse signal measurements.

The display 66 can include any of a number of common display apparatuses and methods. Exemplary display methods can be any combination of a digital or analog display, peak, average or valley pressures, pressure over time as a graph, or other statistic, or an on-off binary indication if certain statistical measures have been met or exceeded.

Pressure waveform signals can be further processed to detect certain specific changes in pressure statistics such as a drop in peak, average or valley pressures and provide a binary user indication.

To further illustrate how the various functional components operate, signal or voltage waveforms from several nodes are illustrated in the graphs described below.

FIGS. 7a and 7b illustrate voltage waveforms of an embodiment of the present disclosure, as shown in FIG. 6. Between nodes A and B, waveform 100v, represents the voltage signal on the guide wire 10, near its proximal end by or at the connector 44 and the electrode or grounding pad 42. This waveform represents a square wave alternating current driven by the power source 64. Because of capacitive coupling losses along the insulated guide wire 10, the voltage plot is not a true square wave, and has a reduced slew rate. This waveform also serves to drive the IC 28. For illustration purposes, this waveform is shown as a 100 KHz current limited and balanced drive signal, however alternative parameters are contemplated as well.

Node C waveform 102 represents the digitized voltage signal used to drive the guide wire 10 from the IC 28 after receiving sensor data. This square wave 102 is then superimposed on the drive signal by the signal driver 206. The resulting signal 100v is seen to incorporate impulses 104. The pressure data from the sensor 26 is encoded in this signal 102 as a time increment 106 in the square wave. The time increment between the impulses 104 corresponds to the width of the square wave in this embodiment. Again, other sensing and logic schemes defining the IC 28 and sensor 26 circuit are contemplated in the presently disclosed embodiments.

The waveforms in FIG. 7a represent those when the sensor 26 is exposed to a lower pressure, while the waveforms in FIG. 7b represent those when the sensor 26 is exposed to a higher pressure. As can be seen the time increment of the square wave and impulse spacing are longer at the higher pressure than at the lower pressure.

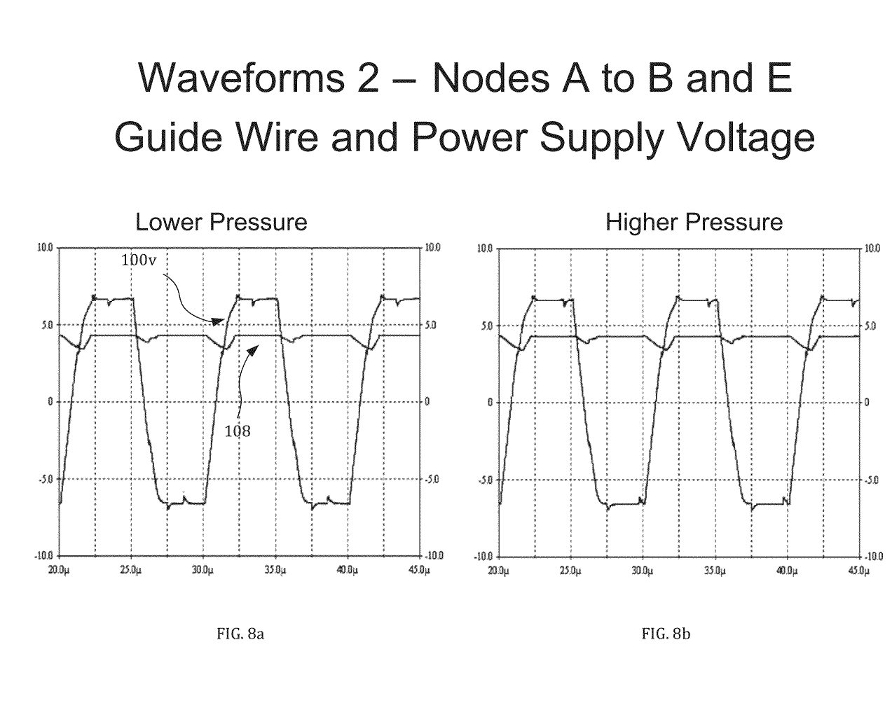

FIGS. 8a and 8b illustrate the IC power supply voltage 108 (power recovery and regulation function 200) at low pressure and high pressure. A drive signal is rectified and filtered to a substantially steady DC voltage, regardless of the pressure applied to the sensor 26. Droops seen in the IC power supply voltage result from drive transitions and are further regulated for smooth sensor DC power.

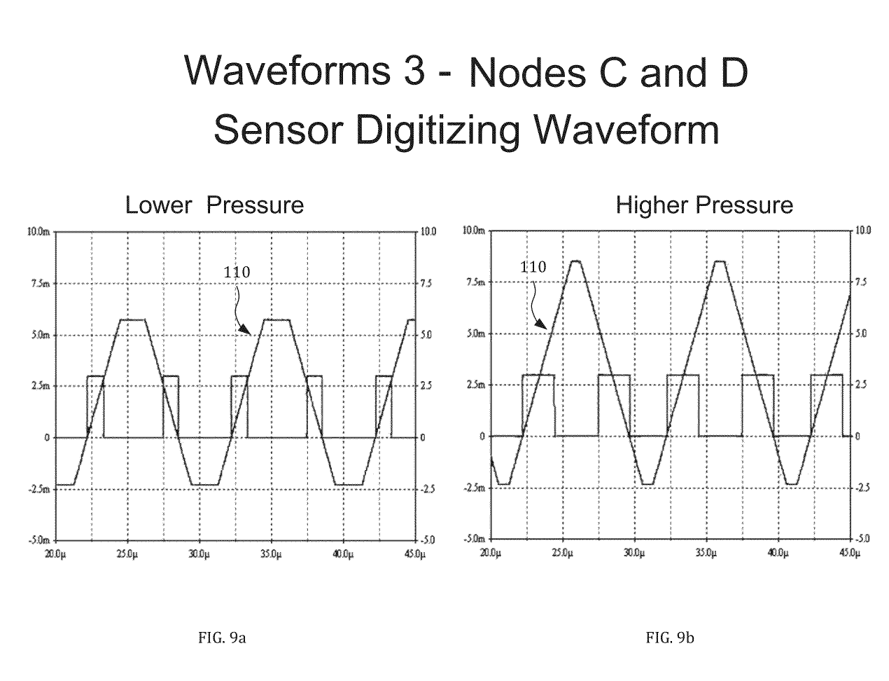

FIGS. 9a and 9b show the interaction between elements of the IC 28 and sensor 26 in one embodiment of the present disclosure. A ramping voltage 110 is applied to the sensor 26 to scan from the voltage imbalance of one arm of the Wheatstone bridge to the voltage on the other. Note the scale for this signal is millivolts. As the voltage applied to comparators is increased, logic gates in the IC 28 determine how large of a resistance imbalance (and therefore pressure) is present within the variable resistance elements of the sensor 26. The greater the resistance imbalance, the higher the voltage ramp 110 must go in order to detect the greater imbalance. Once the imbalance is determined, the square wave 102 signal is returned to zero. The same sequence is applied during the ramp down 110 of the voltage.

As discussed above, the width of the square wave within the IC 28 is applied to the guide wire 10 by the signal driver 206 to encode the pressure sensed by the sensor 26. The width shown in FIG. 9a is lower, due to the lower pressure on the sensor 26, and the width in FIG. 9b is greater at the higher pressure. Other digitization methods of converting pressure signals can be used, but pulse width modulation is show here for illustration purposes.

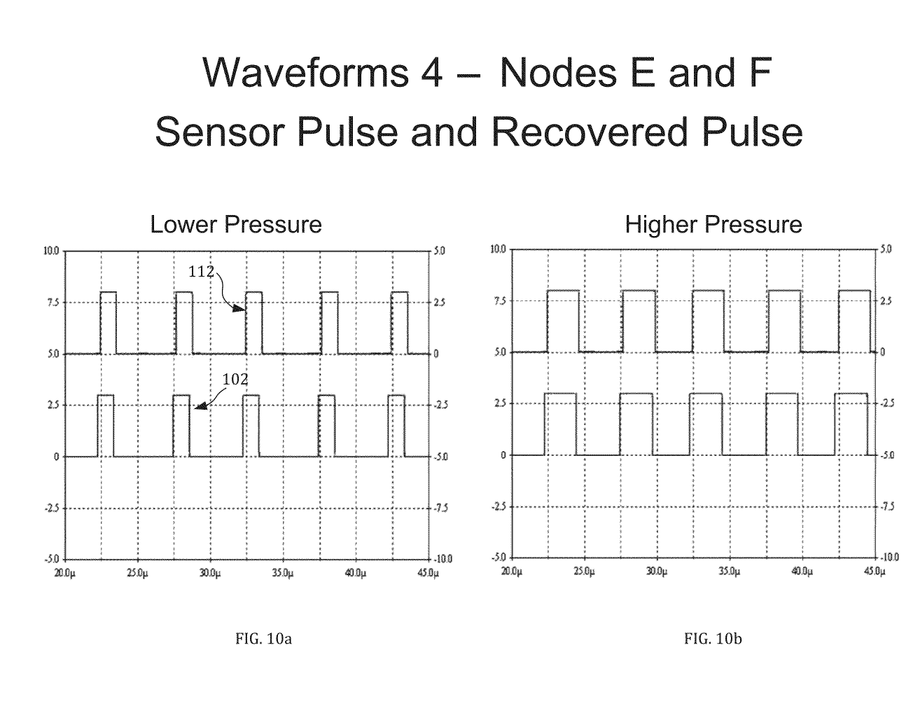

FIGS. 10a and 10b illustrate the waveforms of the square waves 102 generated by the signal driver 206 in the IC 28, and the square waves 112 generated by the signal recovery amplifier 216 in the external readout 212 (which may be part of or operatively coupled to the external power source 64). The scale on the left of the graph is for signal 102, and the scale on the right of the graph is for signal 112. The pulse widths of each are substantially identical in time duration, with only a slight transmission delay. The signal 112 is what goes into the pulse signal processor 218 for conversion to a desirable output, such as a pressure waveform on display 66.

These waveforms demonstrate an embodiment that has an increase in time increment of about 1.1 micro second time for a 0.1% bridge imbalance, which is more than suitable pressure sensitivity for accurate monitoring of blood pressure (certain piezo-resistive sensors exhibit about 2% imbalance at maximum pressure). As with the other electrical components in this guide wire circuit, the system sensitivity can be tailored for specific sensor sensitivity by design.

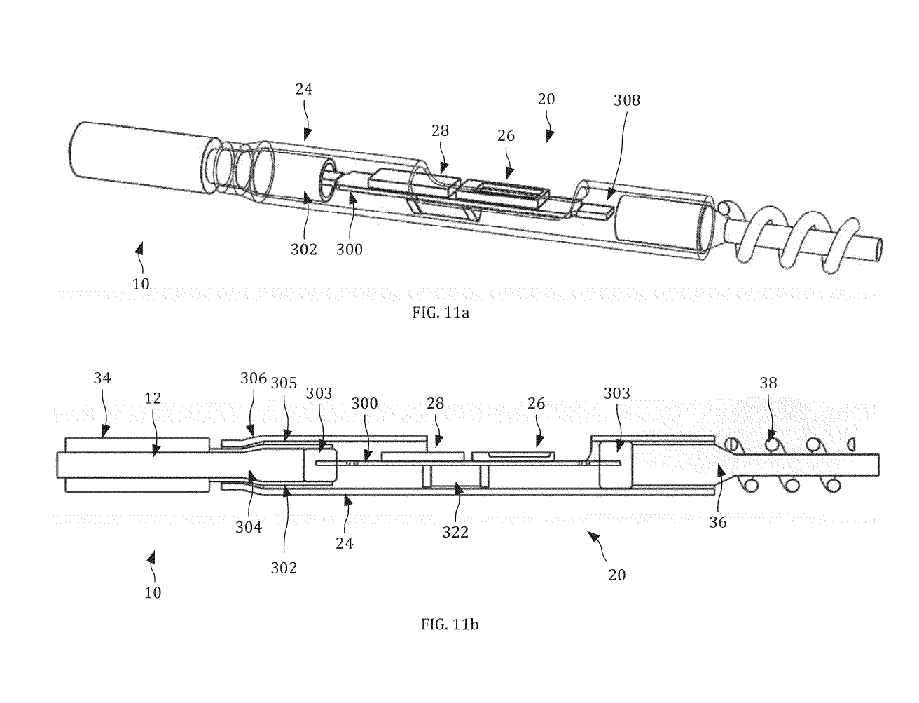

FIGS. 11a and 11b illustrate one embodiment of a distal portion 20 of guide wire 10. Core wire 12 terminates within the proximal end of the sensor housing 24. A portion of the distal end of the core wire 12 may taper 304 up to facilitate a mechanical interlock in a cooperative fashion with a subsequent taper 306 within the proximal end of the sensor housing 24 to enhance the mechanical integrity of the joint therebetween. Other mechanical interlock designs are contemplated as well, such as spiral or circumferential grooves, or other partially or fully interlocking shapes for the core wire 12 and/or the sensor housing 24. Alternatively, a redundant mechanical connection could incorporate the use of a non-conductive tether extending between the core wire 12 and distal core wire 36, bonded by suitable mechanical bonding, such as adhesive.

The core wire 12 conducts electrically with one or more electrical traces (not shown) on a substrate 300 by, for example, a conductive adhesive 303. However, the core wire 12 does not conduct to the sensor housing 24. To facilitate this, a non-conductive proximal insulating sleeve 302 surrounds the core wire 12. Suitable non-electrically conductive adhesive 305 may be used to mechanically join the core wire 12, insulating sleeve 302, and sensor housing 24. Alternatively, the sensor housing 24 could be fabricated of a non-conductive material, such as ceramic, alumina or zirconia, a polymer such as polyimide, PEEK, Vectran, or other rigid polymer, anodized aluminum, oxidized titanium, or similar materials.

Substrate 300 may further include narrowed portions 308 where they are connected to the core wire 12 and distal core wire 36. Narrowed portions 308 serve to mechanically isolate the substrate 300 from mechanical stresses that may be imparted to the guide wire 10.

One or more traces (not shown) electrically connect the distal end of the substrate 300 to the distal core wire 36 by suitable mechanical bonding, such as conductive adhesive, solder, braze, ultrasonic weld, or resistance weld. The mechanical joint at the distal end of the housing 24 may be a solder or braze joint, thereby electrically connecting the distal core wire 36, distal coil 38, and the sensor housing 24. As described above, the relatively small electric current then emanates from these components to the patient to complete the primary circuit 114.

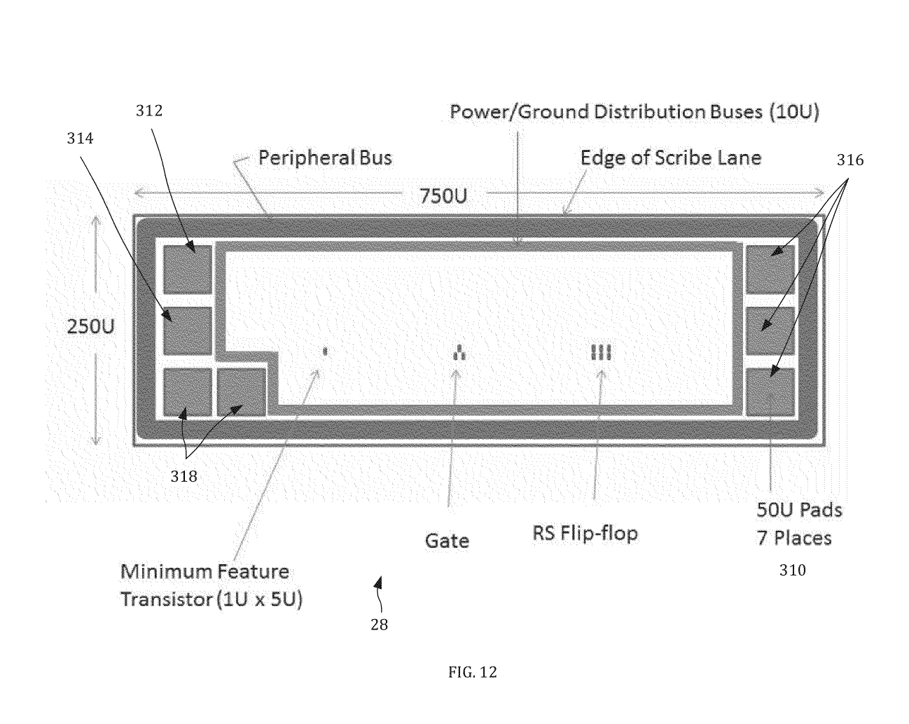

As shown in FIG. 12, integrated circuit 28 is electrically mounted to the substrate 300 by electrical bonding pads 310. IC 28 may include 7 bonding pads 310, or more or less, depending on the electrical functionality desired. Seven bonding pads 310 provide for one power/signal input 312 (from core wire 12 via substrate electrical trace), one power/signal output 314 (to distal core wire 36 via substrate trace), three sensor interfaces 316 (typical for a half-bridge type piezo resistive sensor), and two pads 318 for connection to an optional capacitor 322 that may be mounted on the opposite side of substrate 300. Other exemplary features (e.g. peripheral bus, power/ground distribution buses, transistors, gates, RS flip-flops, etc.) are described in FIG. 12, as might be incorporated into the IC 28, depending on the desired logic scheme desired. Also, typical but merely exemplary dimensions of the IC 28 and the various elements are indicated. Capacitor 322 serves to smooth out the alternating power waveform, thus providing a relatively steady DC current to operate the IC 28. The sensor 26, as mentioned, may have three bonding pads. The electrical connections between the IC 28 and the sensor 26 may be facilitated with electrical bonds to additional traces on the substrate (not shown), or by separate electrical wires therebetween (also not shown).

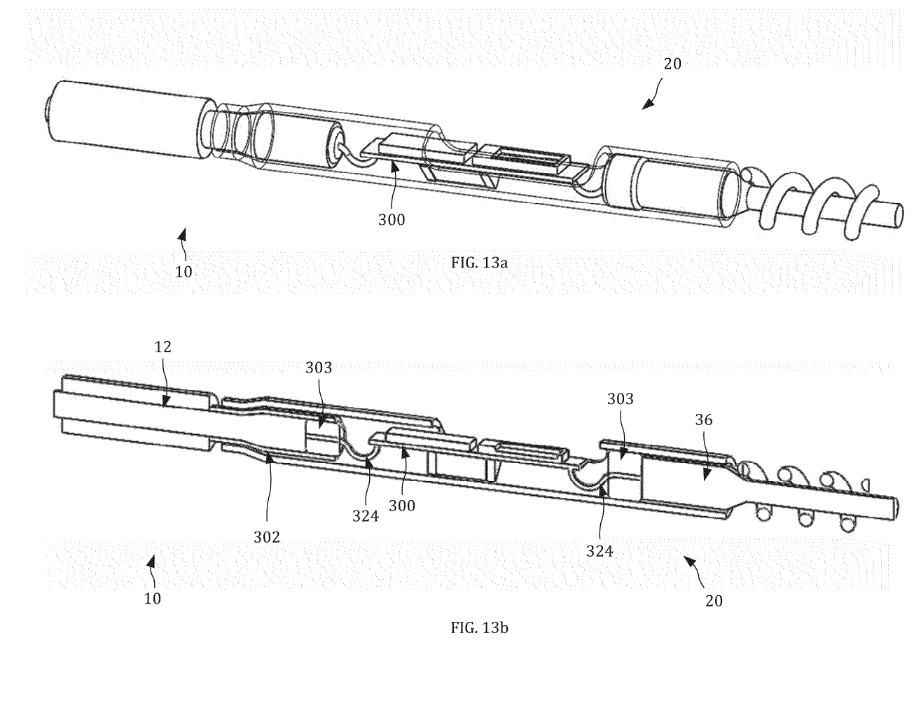

FIGS. 13a and 13b illustrate another embodiment of distal portion 20 of guide wire 10. Here, the substrate 300 may be shorter, so as not to fully extend to the core wire 12 or distal core wire 36. Connector leads 324 electrically connect the core wire 12,via bonding pads on substrate (not shown), to one or more traces (not shown) in the substrate 300, and from the substrate 300 to the distal core wire 36. These connections are relatively short. FIG. 13b also shows where suitable bonding, such as conductive adhesive 303, solder, braze, ultrasonic weld, or resistance weld, could be placed within the proximal insulating sleeve 302, and within a distal interior portion of the sensor housing 24. It should be noted that other embodiments described here may incorporate a conductive joining or bond, and may also make use of the alternatives described previously.

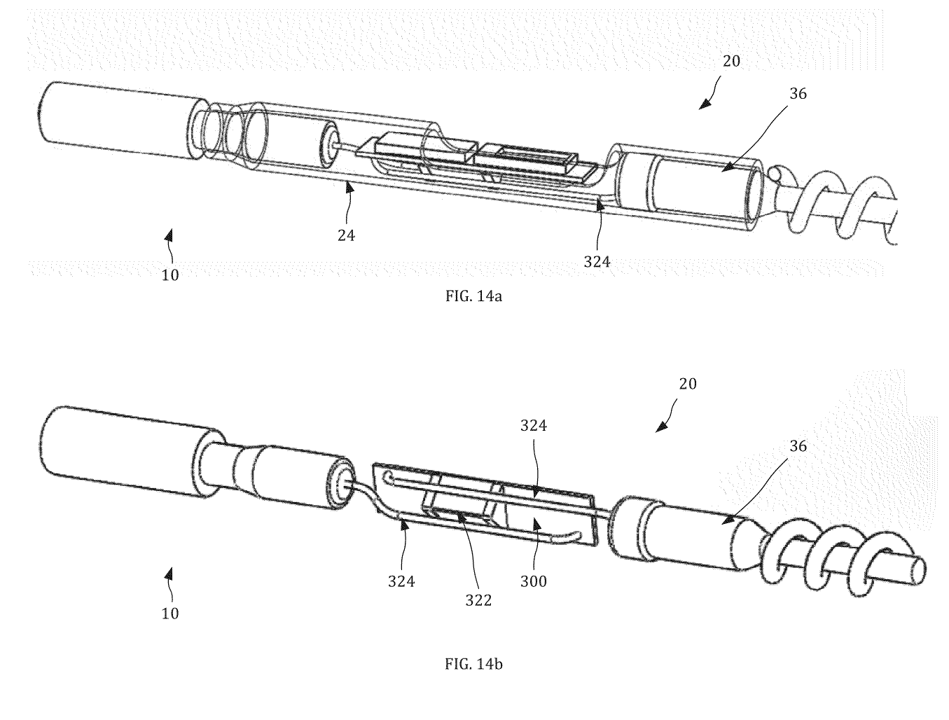

FIG. 14a shows a further embodiment of distal portion 20 of guide wire 10. The electrical connector leads 324 from the core wire 12 and distal core wire 36 are substantially longer than in the embodiment shown in FIG. 13a and FIG. 13b. The connector lead 324 from the core wire 12 may extend to the distal end of the substrate 300, while the connector lead from the distal core wire 36 may extend to the proximal end of the substrate 300. Suitable conductive traces in the substrate may then make the proper electrical connections to the various bonding pads on the IC 28 and sensor 26. It is contemplated that the electrical connector leads 324 can be connected to the substrate 300 at any point along the substrate 300 for purposes of ease of manufacture, assembly, or any reason. FIG. 14b illustrates this same embodiment from below, with the sensor housing 24 removed for clarity.

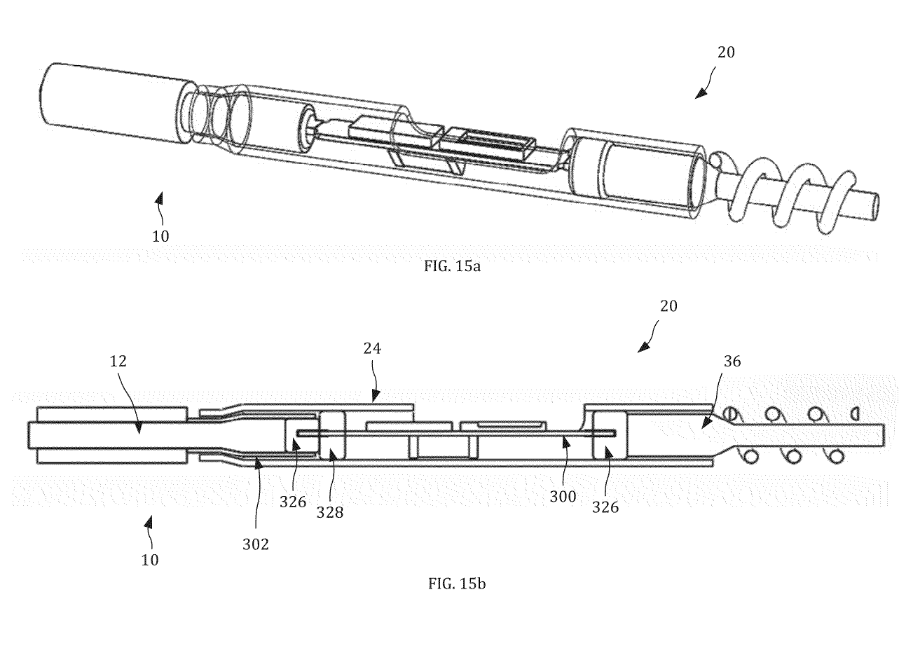

FIGS. 15a and 15b show a further embodiment of distal portion 20 of guide wire 10. The substrate 300, with its electrical traces may be electrically connected to the core wire 12 and distal core wire 36 by a conductive fluid 326, rather than connector leads. A small droplet of conductive fluid 326 adjacent the distal end of the core wire 12, and inside the proximal insulating sleeve 302 near the proximal end of the sensor housing 24, makes this connection on the proximal end of the substrate 300. Examples of conductive fluids include ionic solutions such as saline, conductive hydrogel, or a colloidal solution of metallic particles. Conductive fluids could be either hydrophilic or hydrophobic. A preferred conductive fluid is a silicone fluid loaded with silver particles. A similar droplet adjacent the proximal end of the distal core wire 36 makes the electrical connection for the distal end of the substrate 300. To prevent the distal end of core wire 12 from making electrical contact with the sensor housing 24, a droplet of insulating fluid 328 is placed just distal to the conductive fluid 326. Suitable surface treatments corresponding to the surfaces that contain the conductive and insulating fluid droplets are contemplated to ensure that each droplet stays in its intended location. For example, the proximal insulating sleeve 302 may incorporate a surface that attracts the conductive fluid 326, whereas a portion of the sensor housing 24 may be treated to attract and hold the insulating fluid drop 328. FIG. 15b illustrates this embodiment in cross section.

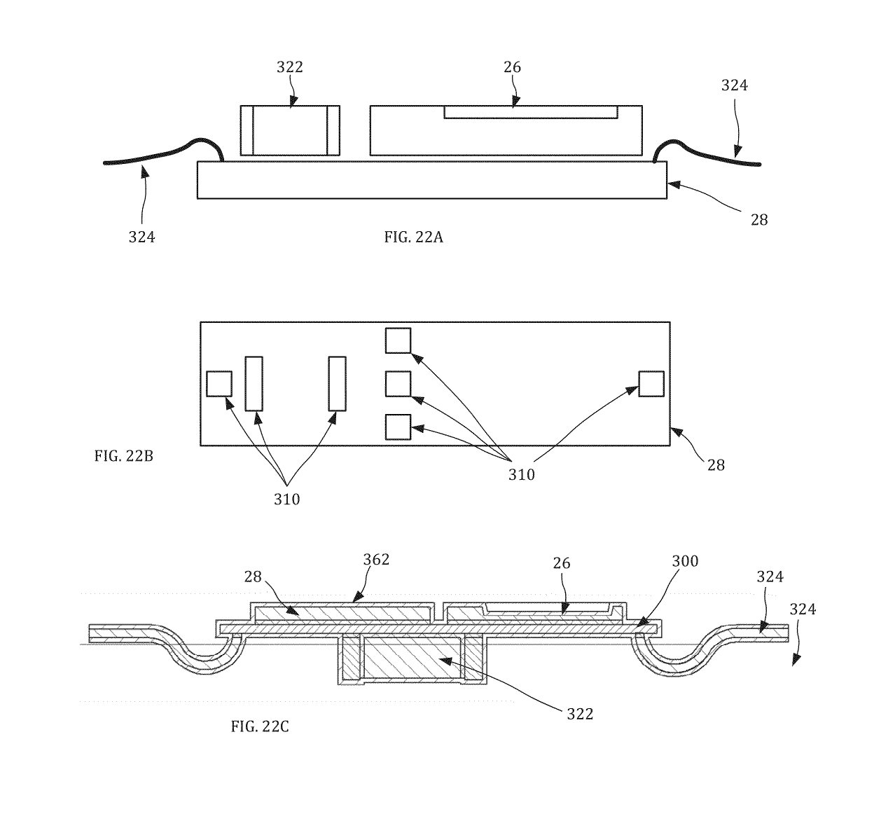

It is also contemplated that the electronic components in the distal portion 20 of guide wire 10 may not require a separate substrate for mounting. As shown in FIG. 22A (side view), the IC 28 may serve as substrate for other electrical components. Here, the capacitor 322 and the sensor 26 are mounted directly to the IC 28. IC 28 is then electrically connected to the core wire 12 and distal core wire 36 by connector leads 324. FIG. 22B is a top view of the IC 28 in such an arrangement, with a number of bond pads 310 for electrical bonding to corresponding electrical components. Other arrangements of the IC 28, sensor 26, and capacitor 322 (if desired) are also contemplated, with or without a separate substrate 300.

It is also contemplated that one or more of the IC 28, sensor 26 or capacitor 322 could be fabricated integrally. For example, the IC 28 and sensor 26 could be fabricated from the same chip.

Any of the contemplated embodiments of the distal portion 20 of guide wire 10 may include an insulating layer 362, as shown in FIG. 22B, covering some or all of the electrical components including, for example, the IC 28, sensor 26, capacitor 322, substrate 300, or the electrical connections of these components to the core wire 12 or distal core wire 36. An example of such an insulating layer is paralene coating.

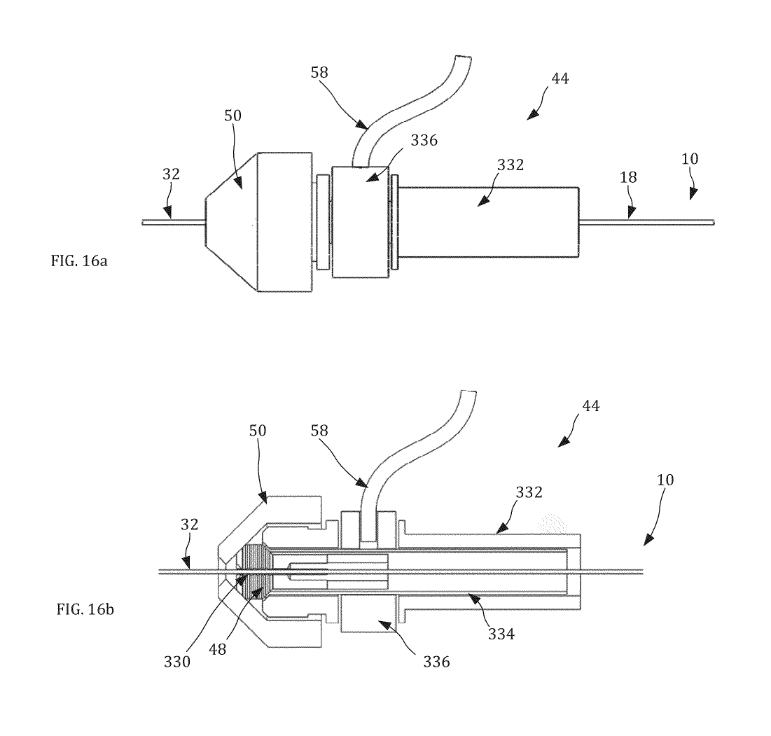

FIGS. 16a and 16b (section view) illustrate an embodiment of an electrical connector 44 which serves to electrically connect the power source 64 to the guide wire 10. Connector 44 may also be used to steer and manipulate the guide wire 10 in the patient. Connector 44 may be secured at any point along the proximal portion 18 of guide wire 10. In this region of the guide wire 10, the core wire 12 may include a lubricious coating 32, which tends to be an electrical insulator. Connector 44 includes a conductive collet or clamp 48 for gripping the guide wire 10. Collet 48 may include sharpened or serrated surface features 330 on inside surfaces to penetrate the lubricious coating 32 and make electrical contact with the core wire 12 beneath. Connector 44 includes a body 332 configured for manipulation by a user's fingers.

Body 332 includes a conductive sleeve 334, which defines an interior lumen. The collet 48 nests inside the distal portion of the conductive sleeve 334. A nut 50 squeezes the collet 48 inwardly when tightened against the body 332. An electrically conductive rotary union 336 may be mounted about an exposed portion of the conductive sleeve 334. Lead wire 58 connects to the rotary union 336. In this manner, rotation and manipulation of the body 332 serves to rotate the guide wire 10, but allows the lead wire 58 to stay in position, not rotating, and thereby not encumbering or becoming entangled with the guide wire 10.

Rotary union 336 may take the form of many known electrical connections which permit relative rotation including, for example, a conductive ball bearing cartridge, or a conductive bushing with one or more brushes extending in to make contact with the conductive sleeve 334. To further facilitate good electrical contact between the rotary union 336 and the conductive sleeve 334, a conductive lubricant may be used.

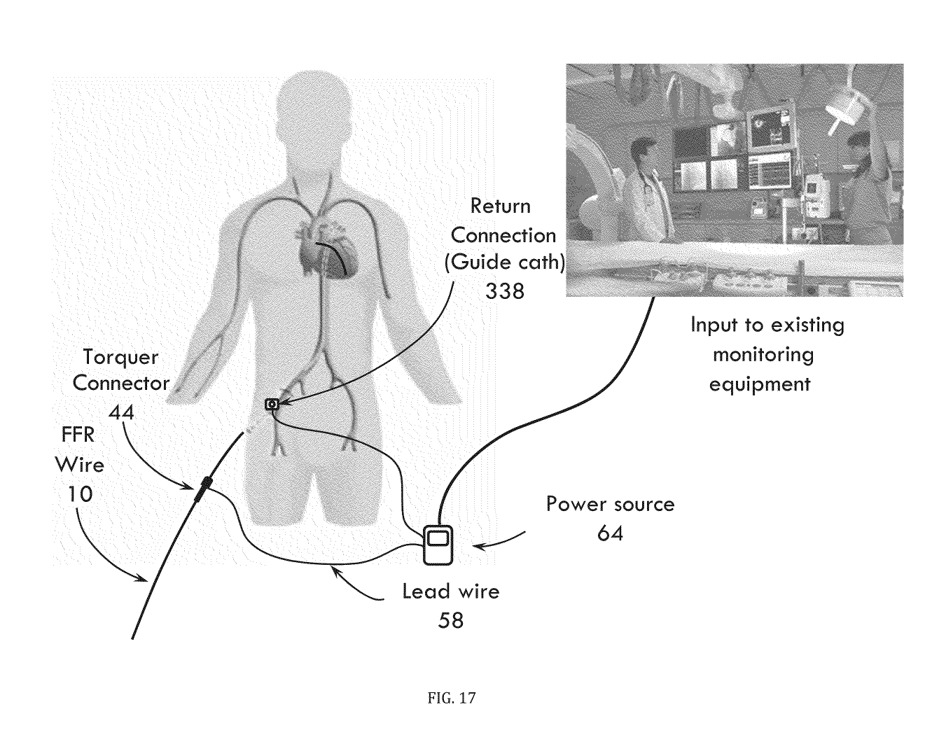

One embodiment of the setup of guide wire 10, as was shown in FIG. 3, makes use of a ground pad 42 for return of the current to the power source 64. An alternative embodiment, illustrated in FIG. 17, makes use of a guide catheter clip 338, to return current to the power source 64. Guide catheter clip 338 is connected to the outside of the guide catheter 62 in an exposed location outside of the patient and near the percutaneous entry point.

Guide catheters are typically constructed using a metallic conductive braid 340 that extends within the walls. This facilitates their torqueability and kink resistance. Guide catheter clip 338 may include a penetration feature to make direct electrical contact with this braid 340.

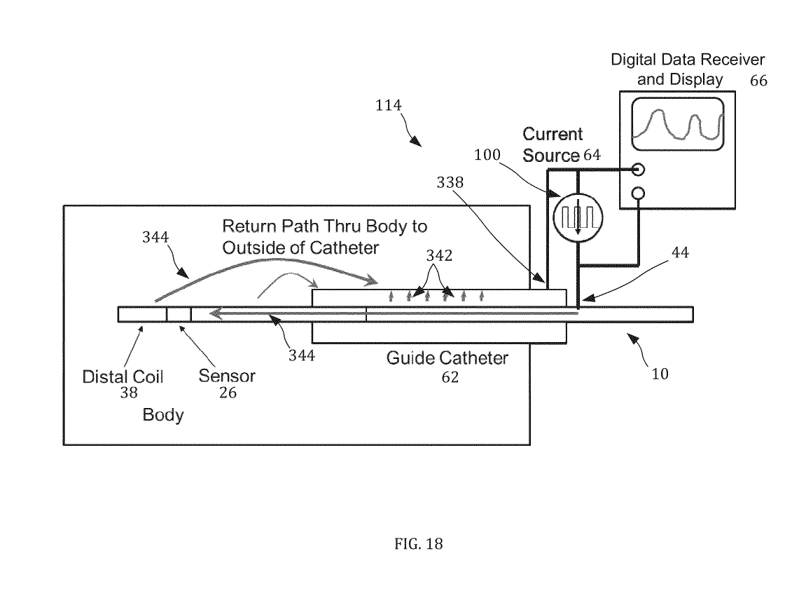

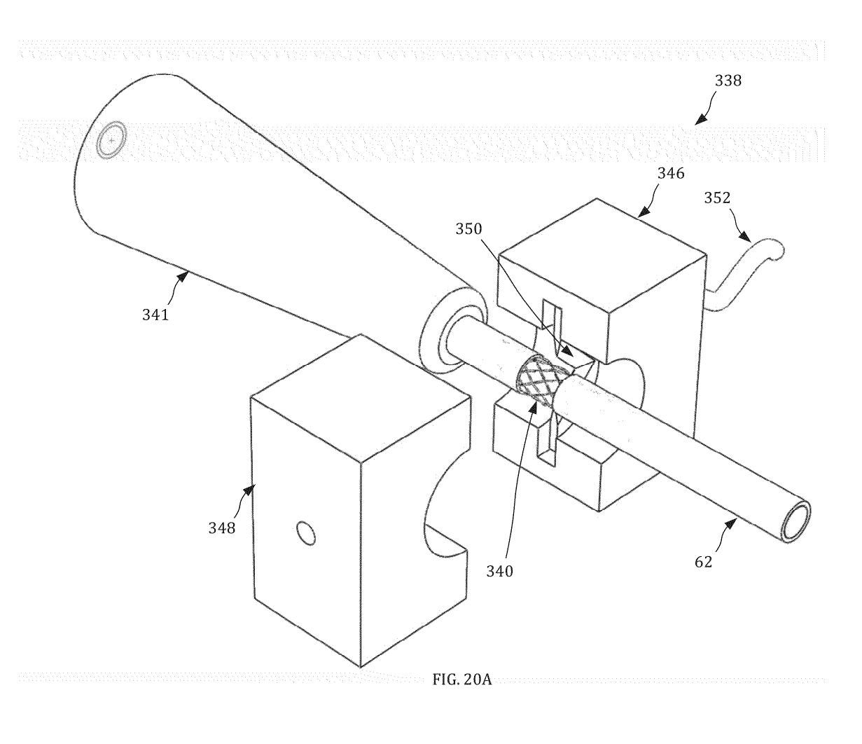

FIG. 18 is a simplified schematic showing the entire circuit (primary circuit 114) of the present embodiment, with a connection 44 to the proximal portion of the guide wire 10, and a direct connection 338 to the guide catheter braid 340 (shown in FIG. 20a), near its proximal end.

The power source 64 which delivers power to the IC 28 may be an alternating drive signal. Alternating signals in excess of 1 kHz may be safer than DC signals of similar amplitude. A preferred signal is 100 kHz. If the drive signal is alternating, the current flow is influenced by capacitive characteristics of the circuit. Therefore much of the current is lost through capacitive leakage from the elongate guide wire 10. Much of this loss current 342 may therefore be contained and not passed through patient tissue if it is collected by the guide catheter braid 340 and brought back to the power source 64 by means of the guide catheter clip 338.

The remaining current (IC current 342) powers the IC 28 and is then passed back to the guide catheter braid 340, mostly to the exterior surface. In this fashion, the RMS current that passes through the patient is greatly reduced.

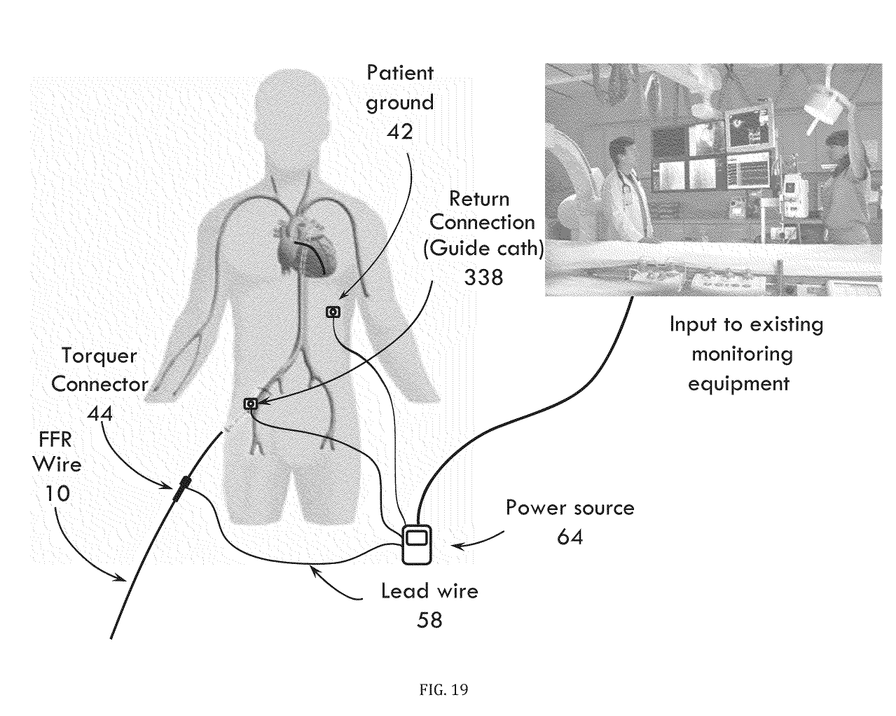

A further alternative embodiment, as shown in FIG. 19, makes use of both a guide catheter clip 338 and a patient ground pad 42. In this embodiment, most of the capacitive loss current 342 is still captured by the guide catheter braid 340, but most of the IC current 344 will travel to the patient ground pad 42. Since the IC current 344 is now in a separate branch of the circuit, it can be monitored. This may provide for additional safety related capabilities, such as shutting the circuit down or modifying the power input in the case of various fault conditions that may impact the current or voltage detected via the patient ground pad 42. Also, normal variations in the patient circuit may be monitored in this branch.

One simplified embodiment of guide catheter clip 338 is shown in FIG. 20A. Clip 338 may be formed of two clamshell pieces 346, 348 that snap together around guide catheter 62. A first clamshell 346 may incorporate a sharpened penetrator 350, configured to penetrate the outer layer of the guide catheter 62 and make direct electrical contact with the braid 340. A clip lead wire 352, connected to the penetrator 350 may be connected to the power source 64. The guide catheter clip 338 is preferably secured around the guide catheter 62 near its proximal end, and just distal to the strain relief 341 if present.

Another embodiment is illustrated in axial section in FIG. 20B, may include V-shaped troughs 354 on the surfaces that contact the guide catheter 62. One or both clamshells 346, 348 may include a sharp penetrator 350 such as a razor blade, preferably oriented at an angle such that as it penetrates the outer surface of the guide catheter 62, it performs a slicing action (here both clamshells incorporate sharp penetrator 350). The slicing action allows the penetrator 350 to cut easily through the relatively soft outer layer of guide catheter 62 until the penetrator just makes contact with the braid 340. A second clamshell 348 may include a spring-loaded bed 356. In this fashion, the guide catheter clip 338 may work with a range of guide catheter sizes. One or more clamshell locks 358 serve to keep the clip 338 in place around the guide catheter 62. Locks 358 may be reversible to facilitate removal of clip 338. Additionally, clip lead wire 352 may be removable. FIG. 20C is an axial section view of the same embodiment of FIG. 20B.

Another embodiment for modulating and encoding (e.g. digitizing) the input waveform is now described. Power source 64 delivers an alternating balanced square wave constant amplitude current 100i, for example at 100 kHz.

IC 28 interrogates the sensor 26 at a particular sample rate, for example 200 samples per second. A circuit in the IC 28 converts each individual pressure data point to a series of digital bits. Each bit serves as input to a variable voltage offset circuit that interacts with the input current waveform 100i to affect the actual voltage drop 100v across the entire primary circuit 114, for a specified period of time, creating amplitude modulation. For example, if a data bit is “0”, the voltage modulation may be reduced for 10 cycles (100vRED), whereas if a data bit is “1”, the voltage modulation is increased for 10 cycles (100vINC). The bits of data may be organized to represent the full value of a given pressure sample.

Additional information may be conveyed in this digital methodology such as sensor temperature or calibration coefficients. One example of a method for conveying digital data that eliminates accumulating DC bias uses “Manchester coding”. The coded data could then be used with the above resistance modulated circuit.

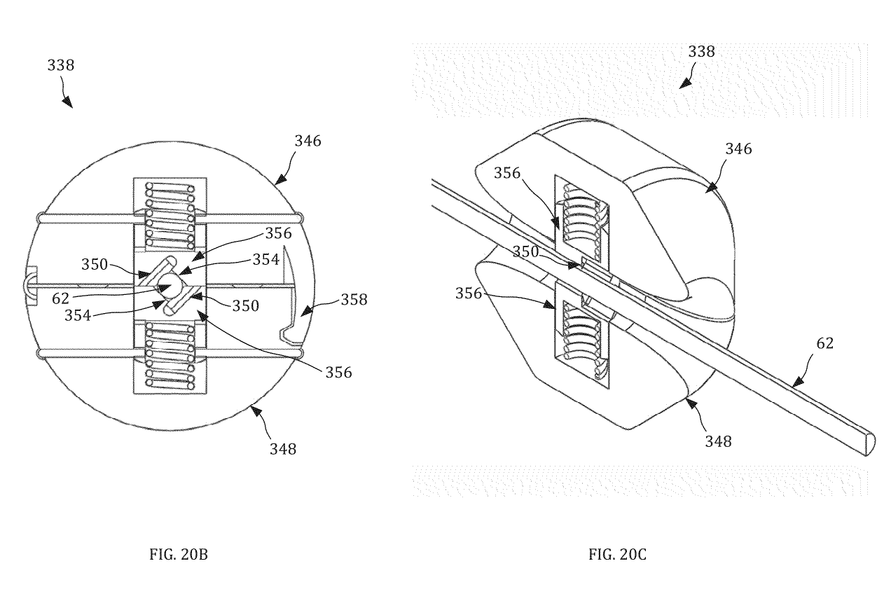

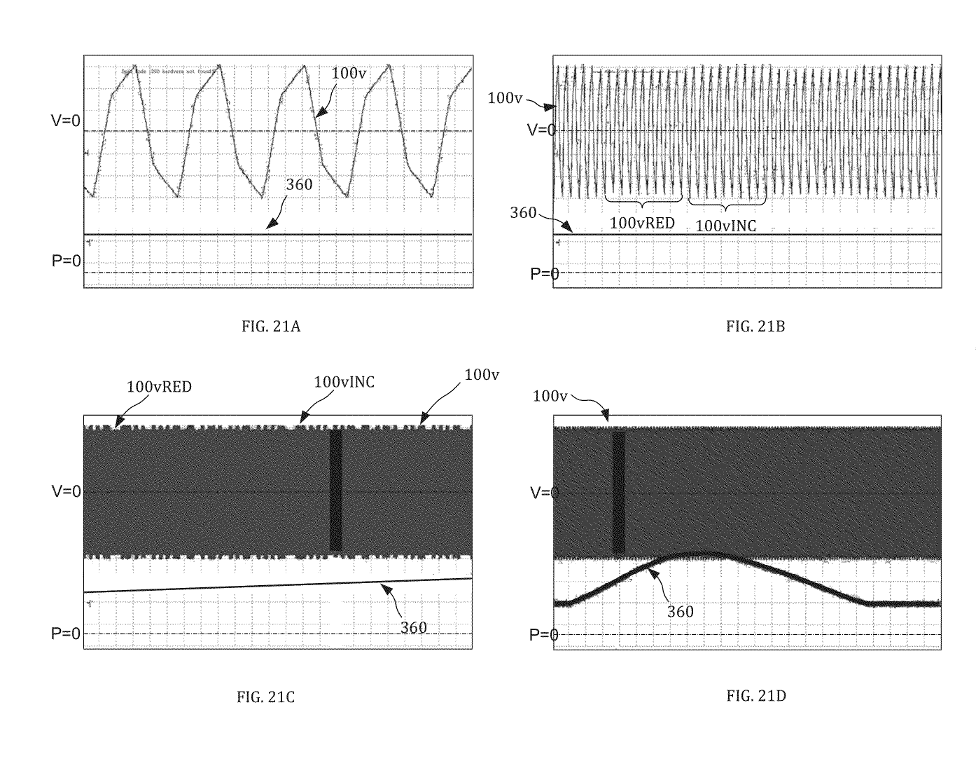

FIGS. 21A-21D serve to further illustrate the resistance modulation and digital encoding with a guide catheter current return path by way of non-limiting example. FIG. 21A graphically shows the primary circuit 114 voltage waveform 100v over several cycles, and the processed pressure waveform 360 as detected by the sensor 26 over a correspondingly short period of time. The zero value line is noted corresponding to the voltage and pressure waveforms. Note that the processed pressure waveform 360 is substantially constant, as the time period shown represents a time interval that may be shorter than the time required for a full pressure sampling. Also, the pressure sampling precedes the processed waveform 360 by a period of time.

FIG. 21B extends the timeframe of the waveforms. Here, the voltage waveform 100v can be seen to change in amplitude, with a number of less modulated cycles 100vRED showing reduced amplitude followed by a number of higher modulated cycles 100vINC showing increased amplitude. The pattern of increased and reduced amplitude modulated cycles essentially forms an information bitstream that represents the value of pressure from the pressure sample. Here again, the pressure waveform 360 is substantially constant, as this is still a relatively short period of time where the processed pressure is still from the prior sampling period. The data bits encoded in the voltage waveform 100v are part of the sampling that will result in the next pressure value to be charted.

FIG. 21C again extends the time of the waveforms. Now, the individual bits of modulated data can be easily discerned as notches on the voltage waveform 100v. And the processed pressure waveform 360 shows a slope, representing the fact that multiple samplings are represented, and the processed pressure waveform shows changing values from the multiple samplings. The pressure waveform may interpolate or smoothen the sampled pressure data points, as shown here. This results in a smooth waveform rather than a stairstepped waveform.

FIG. 21D represents yet a longer timeframe, such as the time of a full heartbeat. The full pressure pulse of the heart is able to be represented by the amplitude modulation digital encoding scheme as described here. Such a scheme is highly resistant to errors introduced by ambient sources of electrical noise such as fluorescent lights, x-ray imaging equipment, radio telemetry devices, or other electronic devices that may be in proximity to the guide wire 10 during use.

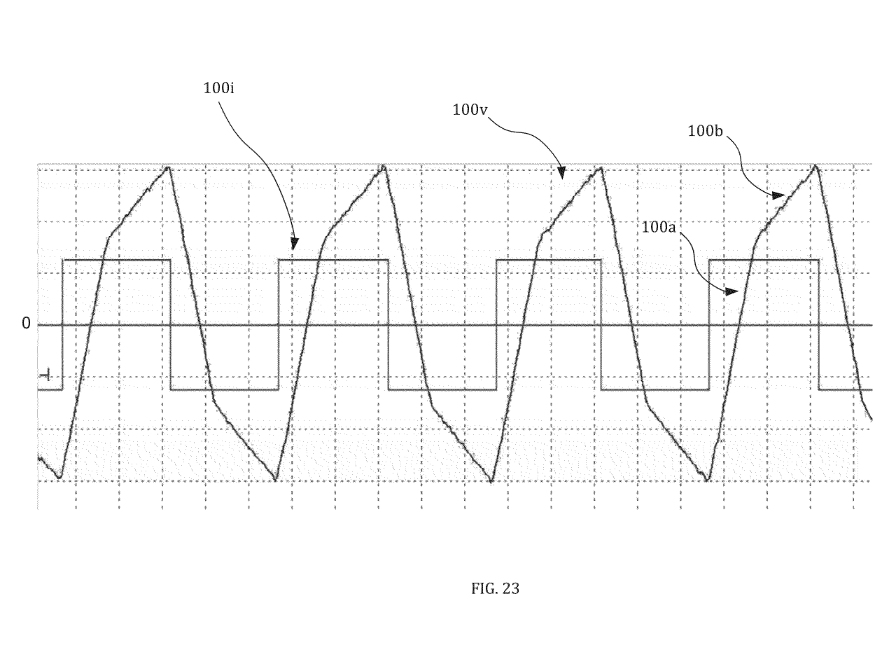

Referring back to the exemplary primary circuit 114 voltage waveform 100v as described in FIG. 21A and seen in FIG. 23, the resultant voltage of balanced square wave constant current input 100i is shown in FIG. 23. The resulting voltage waveform 100v reflects capacitance and other effects in the full primary circuit 114. The first slope 100a of the voltage waveform 100v, in this example, is due primarily to the capacitance of the guide wire 10 formed with the interior of the guide catheter (and creating the loss current 342). Once the voltage reaches a certain level, in this case after a transition of about 9 volts (peak to peak), the IC 28 circuit is energized, allowing current to flow through the IC 28, to charge the capacitor 322. The second slope 100b represents additional capacitive effects from the exposed distal portion 20 of the guide wire 10. This latter current is essentially operating current 344 for the IC 28. This IC current 344 is leaked to the patient. This current 344 may be over and above what is needed to power the IC 28, due to the additional capacitive effect seen in slope 100b.

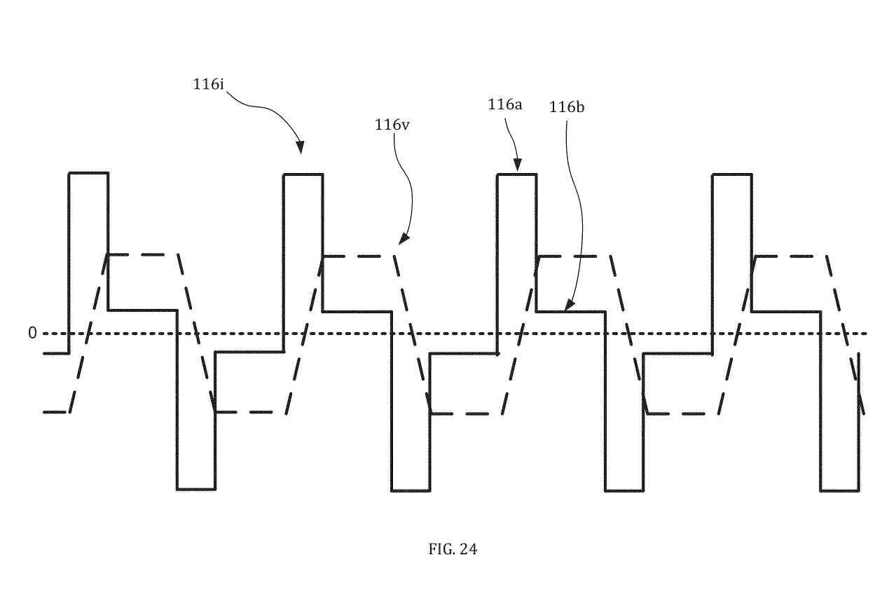

To further minimize current that flows through the patient (beyond the use of the guide catheter braid 340), various alternative drive signals are also contemplated, beyond the balanced current square wave 100i previously described. One alternative is a stepped square wave current 116i, as shown in FIG. 24. A first portion of stepped square wave 116a has a higher amplitude, for purposes of quickly ramping up the capacitive charging between the guide wire 10 and guide catheter 62. But thereafter the current decreases significantly to a second portion of stepped square wave 116b. This portion of the waveform is substantially less than that in FIG. 23. The result of the stepped square wave current is a lower voltage waveform 116v (shown here as a dashed line). An RMS calculated current within the patient is thus substantially lowered with this type of stepped square wave current drive.

Other ways to minimize current delivered to the patient are also contemplated. For example, the IC 28 may be configured to operate on a low voltage, for example 1 to 2 volts, or even lower, in contrast with the 4.5 volt (9 volt peak to peak) example described above. The IC 28 may be configured to operate at any nominal voltage, the lower the operating voltage, the lower the required current to power it because less time is spent changing charge polarity of the guide wire 10 within the guide catheter 62.

Many of the embodiments of guide wire 10 contemplated include a core wire 12 which is of a single material, for example stainless steel, or more specifically cold worked type 304 stainless steel. However alternate materials are also contemplated, such as other stainless steels, nickel titanium alloy, cobalt chromium alloys, or other materials that exhibit high strength and high resilience. Some of the core wire 12 may be fabricated of one material, while some may be fabricated from another material. For example, the proximal portion 18 may be stainless steel, while the intermediate portion 22 may be fabricated from nickel titanium alloy, using suitable joining techniques. Also some or all of the core wire could be fabricated from a composite material, such as carbon fiber/epoxy or drawn-filled tube.

The distal core wire 36 may be in the form of a ribbon, or may be generally circular in cross section for a portion of its length, and generally rectangular for a portion of its length, or may be generally circular for its entire length. Distal core wire 36 may also have one or more tapering diameter sections. While it may be preferable to form the distal core wire 36 of 304 stainless steel, other materials are contemplated as well, such as nickel titanium alloy, cobalt chromium alloys, other 300 or 400 series stainless steel, other spring steels, or other suitable materials.

The distal core wire 36 may have multiple components, for example a circular wire extending alongside a ribbon, each one extending for a portion or fully through the distal portion from the sensor housing 24 to the distal tip 40. Other structures are contemplated as well, for example a braid, a twisted ribbon, a hollow cable, or a solid cable, or combinations of these or other components described previously.

Other embodiments of the present disclosure will be apparent to those skilled in the art from consideration of the specification and practice of the features disclosed herein. Also, features and embodiments of the present disclosure may be used separately or in any suitable combination. Further, while the specification describes certain details of the present disclosure to illustrate various embodiments and features, it is intended that the specification be considered as exemplary only, with a true scope and spirit of the disclosure being indicated by the following claims.

1. An elongate intravascular guide wire, comprising:
an electrically conductive core wire connected to a sensor housing;
a pressure sensor and an integrated circuit located within the sensor housing;
at least one return electrode connected to the integrated circuit, wherein the integrated circuit, pressure sensor, and return electrode are electrically connected to form part of a primary electrical circuit; and
an electrical connector disposed on a proximal portion of the core wire, and electrically connected to the core wire, said core wire being configured to deliver electrical power to the integrated circuit from the connector, and said integrated circuit being configured to transmit electrical information.

2. The guide wire of claim 1, wherein the integrated circuit is configured to convert transduced pressure information from the pressure sensor to digital information, and convey the digital information to the primary electrical circuit.

3. The guide wire of claim 1, wherein the core wire is solid.

4. The guide wire of claim 1, wherein at least a portion of the core wire comprises stainless steel.

5. The guide wire of claim 1, further comprising a distal coil extending from the sensor housing, wherein the distal coil forms at least part of the return electrode.

6. The guide wire of claim 1, further comprising a proximal coil extending from the sensor housing, wherein the proximal coil forms at least part of the return electrode.

7. The guide wire of claim 1, further comprising a lubricious coating extending along the core wire to electrically insulate the core wire.

8. The guide wire of claim 1, wherein said connector further comprises an electrically conductive clamp.

9. The guide wire of claim 8, wherein said clamp is configured to establish electrical contact with the core wire.

10. The guide wire of claim 8, wherein said clamp is capacitively coupled to the core wire.

11. The guide wire of claim 1, wherein the core wire is hollow.

12. A system for measuring intravascular blood pressure within a mammal, comprising:
an elongate guide wire including:
a core wire,
a pressure sensor at a distal portion of the core wire, the pressure sensor being configured to sense blood pressure, and
an integrated circuit at the distal portion of the core wire, the integrated circuit being electrically connected to the core wire;
a steering device in electrical communication with the core wire;
a power source connected to the steering device;
a guiding catheter surrounding at least a portion of the guide wire; and
wherein the integrated circuit operates with the pressure sensor to convert the sensed pressure to an information encoded signal sent along the guide wire.

13. The system of claim 12, further comprising an external grounding pad configured to be connected to the mammal.

14. The system of claim 12, wherein the pressure sensor is a variable resistance sensor.

15. The system of claim 12, wherein the pressure sensor is a capacitive sensor.

16. (canceled)

17. A method for measuring blood pressure in a patient comprising:
advancing a guide wire to a desired position in the patient's blood vessel, the guide wire including a pressure sensor and an integrated circuit at a distal portion of the guide wire, said integrated circuit being configured to send an information encoded signal representative of blood pressure through the guide wire; and
operating a power source, electrically connected to a connector in electrical communication with the guide wire, to send electrical current along the guide wire; and
measuring the pressure at the desired position.

18. The method of claim 17, wherein advancing the guide wire to the desired position includes advancing the guide wire to a desired position in the patient's coronary blood vessel, and measuring the pressure includes measuring blood pressure at the desired position in the coronary blood vessel.

19. The method of claim 18, further including comparing blood pressure in the coronary blood vessel to aortic blood pressure.

20. The method of claim 17, wherein advancing the guide wire to the desired position includes advancing the guide wire to a position downstream of a constriction, and measuring the pressure includes measuring blood pressure at the downstream position.

21. The method of claim 20, further including determining a fractional flow reserve based on the blood pressure at a position upstream of the constriction and the blood pressure at the downstream position.

22-49. (canceled)
